(19) United States
(12) Patent Application Publication
(10) Pub. No.: US 2024/0148322 A1
(43) Pub. Date: May 9, 2024

(54) MONITORING CHANGES IN VOLUME AND PROPERTIES OF HUMAN INTERSTITIAL FLUID AND ELECTRO-MECHANICAL DESIGN OF A MAGNETIC DEVICE TO ACCOMPLISH THE SAME

(71) Applicant: BodiGuide, Inc., Bellevue, WA (US)

(72) Inventors: William Oren WEKELL, Maple Valley, WA (US); James P. GAMACHE, Bellevue, WA (US); Deborah A. KESSLER, Bellevue, WA (US)

(21) Appl. No.: 18/369,123

(22) Filed: Sep. 15, 2023

Related U.S. Application Data
(60) Provisional application No. 63/407,039, filed on Sep. 15, 2022.

(51) Int. Cl. A61B 5/00; A61B 5/107

(52) U.S. Cl. CPC A61B 5/4878; A61B 5/1073; A61B 5/4839; A61B 5/6831; A61B 5/6843; A61B 2560/0209; A61B 2562/0223

(57) ABSTRACT

In a wearable device and methods of monitoring changes in the volume and properties of human interstitial fluid associated with both normal and disease conditions using limb circumference and acceleration, raw data is transmitted from the wearable device to external devices for storage and evaluation. The data is compared and contrasted with reference information derived for the individual as well as a larger population of individuals and evaluated for wellness and clinical implications. Related messages regarding the state of the individual may be transmitted to concerned parties such as patients, caregivers and medical professionals.

### BACKGROUND

Fluid balance is a significant aspect of human physiology. The body automatically makes adjustments in an attempt to maintain the body's fluid levels under a variety of conditions. Certain medical conditions such as heart failure, kidney disease, and others may exceed the body's regulatory mechanisms, resulting in excess fluid retention or fluid loading, for example, which may present as peripheral edema or limb swelling.

### SUMMARY

Fluid retention and edema, or limb swelling, can be associated with and be predictive of an impending decompensation event for heart failure patients. Unfortunately, patients generally do not have good tools for recognizing markers of deteriorating condition. This often forces the patient to the hospital for intervention. This is frightening, expensive, and can be life threatening.

Measuring and managing fluid balance is associated with many aspects of human health such as heart health, any number of disease states and medication intervention trials which could benefit from an improved wearable device that can detect early warning signs of a new or worsening medical condition in fields that may include, without limitation, nephrology, cardiology, sports medicine, prenatal care, migraines, drug trials, and the like.

### BRIEF DESCRIPTION OF THE DRAWINGS

The detailed description is described with reference to the accompanying figures that illustrate aspects of one or more embodiments described herein, in which the left-most digit(s) of a reference number identifies the figure in which the reference number first appears. The use of the same reference numbers in different figures indicates similar or identical items.

FIG. 1 shows an isometric view of a device as an example that can be configured to monitor changes in volume and properties of human interstitial fluid.

FIG. 2 shows an exploded view of an example measurement assembly, winder cassette, strap, and clasp of a device corresponding to the example illustrated in FIG. 1.

FIG. 3 shows an exploded view of the winder cassette, strap and clasp that remains when the measurement assembly is removed in some embodiments.

FIG. 4 is a top view of the winder cassette relative to the strap and clasp.

FIG. 5 is the partial cross section view A-A in FIG. 4 of the winder cassette in the assembled condition.

FIG. 6 is a side view of the device, which may correspond to the device illustrated in FIG. 1.

FIG. 7 is a detail section view through the measurement assembly, winder cassette, strap and clasp of the example device illustrated in FIG. 6, taken along line B-B.

FIG. 8 is an isometric view of the device, which may correspond to the device illustrated in FIG. 1 with the enclosure of the measurement assembly removed.

FIG. 9 shows a plot of normal swelling (e.g., swelling of a limb) over a time period of 12 days for a hypothetical heart failure patient.

FIG. 10 shows an 8-day plot of a compensated swelling event (e.g., swelling of an ankle) associated with fluid retention related to increased salt intake for a hypothetical heart failure patient.

FIG. 11 shows a plot of a hypothetical decompensated fluid retention episode such as one preceding a heart failure hospitalization.

FIG. 12 shows an example of a loop that illustrates the efficacy of integrating interstitial fluid volume monitoring via the device with a determined outcome.

FIG. 13 illustrates an example of an integrated medication treatment log and anklet circumference history.

FIG. 14 illustrates an example architecture to implement monitoring changes in volume and properties of human interstitial fluid in a patient.

FIG. 15 illustrates various components of a measurement assembly arranged in accordance with one or more embodiments described herein.

FIG. 16 illustrates an example of the database server shown in FIG. 12.

FIG. 17 is a flow diagram of an example process that may be performed at least in part by a measurement assembly for measuring limb circumference of a subject, generating a waveform of the measurements over time, and outputting indication of a condition exposed by the waveform.

FIG. 18 shows an isometric view of a device as another example that can be configured to monitor changes in volume and properties of human interstitial fluid.

FIG. 19 shows an exploded view of an example measurement assembly, winder cassette, strap, and clasp of a device corresponding to the example illustrated in FIG. 18.

FIG. 20 shows an exploded view of the winder cassette, strap, and clasp that remains when the measurement assembly is removed in some embodiments.

FIG. 21 illustrates an example of a digital circuit that may be implemented to perform quadrature decoding including counting revolutions (a digital pass through a 0 degree reference angle) detection in software at a fraction of the power used by some microprocessor quadrature decoder circuits.

FIG. 22 is a top view of the winder cassette relative to the strap and clasp.

FIG. 23 is the partial cross section view A-A in FIG. 22 of the winder cassette in the assembled condition.

FIG. 24 is a side view of the device, which may correspond to the device illustrated in FIG. 18.

FIG. 25 is a detail section view through the measurement assembly, winder cassette, strap, and clasp of the example device illustrated in FIG. 24, taken along line B-B.

FIG. 26 is an isometric view of the device, which may correspond to the device illustrated in FIG. 18 with the enclosure of the measurement assembly removed.

### DETAILED DESCRIPTION

Interstitial fluid volume is a key medical concern in patients with conditions such as heart failure decompensation, reduced kidney function, and similar physiological conditions. In an example of particular concern, increases in interstitial fluid volume are associated with heart failure decompensation. Taking singular measurements from time to time, perhaps at doctor's appointments, can yield dramatically different and contradictory results from measurement to measurement, depending on the random time of day of the appointment and other factors. Absent presumably more sophisticated measurements, doctors often resort to gross approximation of the presence or absence of swelling, such as pressing their finger into one's ankle, as equivalent in efficacy. Moreover, lacking practical and rigorous medical measurement has constrained the usefulness of this physiological parameter.

Previous approaches to circumference monitoring of limbs have focused on measuring the circumference at a known position on the limb. The device and techniques described herein are not dependent on a known position on the limb but rather take measurements at the minimum circumference regardless of where it occurs as indicating a measure of interstitial fluid volume.

In some embodiments, the device includes a strap constructed to impart only a light tension of the strap around the limb, achievable by design including one or more of lightweight components, the material characteristics of the strap, and the width of the strap. These parameters, as well as the activity of the limb, may all combine to allow the device to come to rest at the minimum circumference of the limb such that the device is continuously sensing the minimum circumference of the limb without unnecessary pressure around the limb at the point of measurement. For example, when the device is used to measure the wrist or ankle volume, it settles at or near the minimum circumference of the limb such that measurements may be compared over time. It may also settle to a different repeatable home location because of the construction details. Movement of the limb (such as standing or walking) helps facilitate device mobility on the limb.

The device, in addition to moving along the axis of the limb, can rotate about the axis of the limb. In general, the limb is not circular in cross section. As it clocks about this circumference the area of the winder cassette that bears against the limb has been minimized and the strap can fit more closely to the actual shape of the limb. Thus, the device may allow for consistent measurements regardless of the clocking orientation of the device about the axis of the limb.

The device described herein may be configured to measure limb circumference and acceleration on an intermittent, periodic, and/or continuous basis, and to analyze the circumference measurements and the device and/or subject orientation including comparing the current state or trends with the patient's baseline, normal, or desired state. Differences between measured states and desired states may be used as input to guide decisions about the subject's diet, activity, and treatment, including medication in some implementations, to advance the subject toward the desired state and avoid potentially life-threatening events such as breathless hospitalizations for heart failure patients, in addition to other events such as those described herein.

One aspect of the device may comprise a low force tensioning element and may be configured to include a large area of distributed load using, e.g., a lightweight strap (in one example, less than ½ ounce, inclusive of the device) to allow even force distribution. Gravity acting on the mass of the device may contribute force. An additional force is contributed by the mechanism that holds the position device in intimate contact to the limb. For example, tension against the subject's skin exerted by the device, and particularly by the strap under tension, may be less than or substantially equal to (there may be some light compression of the skin) the interstitial fluid pressure at the site and distributed across the surface of the strap such that friction against the skin balances against the force of gravity on the weight of the device. These in conjunction represent the combined forces exerted over a prescribed area. One embodiment of the device may provide a broad surface of approximately 25 millimeters in width disposed around the limb to distribute approximately 11 grams gravitational load applied along the axis of the limb and the constant spring force load of approximately 10-40 grams snugging the strap to the limb.

In at least one embodiment, the disclosed device measures circumference by means of a magnetic circuit and a strap encircling the limb. The magnetic circuit includes or cooperates with a magnet and a magnetic sensor. As the limb changes size, the strap expands and contracts via a strap housing and tensioning mechanism which allows the magnet to rotate and the magnetic sensor to detect the angle of rotation of a spool associated with the increasing and decreasing circumference of the limb.

For example, when a person is lying down, interstitial fluid in the body distributes itself somewhat evenly about the length of the body. But when a person is standing or sitting in a relatively vertical position, fluid is redistributed to the limbs due to gravity. This effect is more pronounced in the lower extremities.

In one embodiment, the device may be worn near continuously and circumference and orientation data are collected at a specified interval and processed to determine a Daily Swelling Pattern and Average Daily Swelling for each individual. The intraday circumference changes related to the redistribution of interstitial fluid from lying to standing is greater than the change in circumference associated with fluid retention of interest associated with health or medical conditions that require attention. Therefore, a plurality of measurements of circumference over circadian cycles can be used to detect longitudinal fluid retention.

Careful quantification and tracking over time show that even changes averaging only a few millimeters in fluid volume, which are pertinent physiological information, can present as a significant sign of disease progression but be invisible at a single inspection, or sometimes even daily inspection, and indicate more precise measurement than is possible by visual inspection alone. Capturing a plurality of measurements of minimum limb circumference typical of a narrowing of the limb at an ankle or wrist can be beneficial, as limb circumference can be a close proxy of interstitial fluid volume.

The device may also measure other physiological parameters such as heart rate, peripheral capillary oxygen saturation (SpO2), respiration rate, and non-invasive blood pressure (NIBP). By means of emitting various frequencies of light and evaluating the returned energy, it is possible to evaluate a number of physiological phenomena. Using green light, for example, to sense heart rate in conjunction with measuring for peripheral edema, it is possible to corroborate the finding of the swelling event and improve the certainty and more accurately evaluate progression of a decompensation event. Similarly, using, for example, red and infrared light to evaluate blood oxygenation in conjunction with a stress test, for example, can provide valuable information on the current limitations of the oxygen transport system. And this can further corroborate and calibrate the peripheral edema measurements and appropriately scale the response to the patient's changing condition. The use of optical measurements to evaluate noninvasive blood pressure in the patient is also envisioned.

Noninvasive blood pressure is used in the definition and adjustment of the patient treatment including the selection and dosing of medications. Physiological parameters may be collected on separate devices such as weight using a scale, or NIBP using a device including an inflatable blood pressure cuff, and utilized in the analysis of the patient condition.

The monitoring, especially continuous monitoring, of a physiological parameter on a remote device such as the one described here may call for aligning the time of data collection by the device with an established time standard such as Universal Time Coordinated (UTC) external to the device, where the data might be stored and analyzed other than on the device. The device may calculate a relative time such as the time from initial boot as the time the measurement is taken, which can then be resolved as an actual time when utilized with external devices that are synchronized with universal time standards to determine the time of measurement with sufficient accuracy for the planned use. This method may eliminate the need to perform at least some clock synchronizations on the device at startup and during use, thereby reducing operational complexity and power requirements when compared to synchronizing the real-time clock on the device using other time keeping methods. To integrate the treatment plan with the patient outcome, medication logs may be collected to associate modifications in the treatment plan with the patient outcome.

In some embodiments, power requirements may be minimized by keeping the device (specifically, its processor) in sleep mode for much of the time. Using timers and interrupt circuits, the device can still respond to programmed and real time events, and awaken appropriately when required. In at least one embodiment to this end, an angle sense component is connected to a comparator to produce an interrupt at 0 and 180 degrees rotation of the spool. The rate of interrupts associated with a rapid extension or retraction of the strap may be used to wake up the processor. By comparison with a processor-integrated or external quadrature detection that stays energized, dramatic power savings can be achieved, extending battery life. Additionally, this interrupt circuit can be used to trigger the processor to go into Bluetooth advertising mode or other communication protocols, or other variable sections of software. In some embodiments of this device, the accelerometer may be used as an interrupt source in order to trigger additional actions by the processor such as initiating communication protocols or other variable sections of software in response to detecting a change in position or orientation.

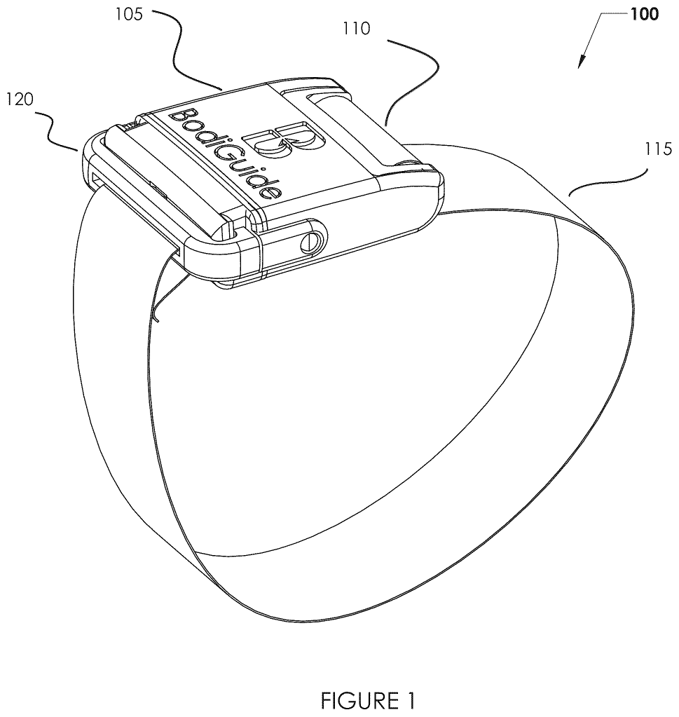

FIG. 1 shows an isometric view of a device 100 as an example that can be configured to monitor changes in volume and properties of human interstitial fluid. The device 100 may include, among other components, a device assembly comprising a measurement assembly 105, a winder cassette 110, a strap 115 that is configured to be placed around the limb of the subject, and a clasp 120. In at least some embodiments, the subject may wear the device 100 around the lower arm (i.e., below the elbow) or lower leg (i.e., below the knee), although the teachings herein may apply to other body parts, especially body parts that have axial portions, and thus should not be considered limited except by the type of needed data and where the data might be obtained.

The measurement assembly 105 may house an electronics subassembly to obtain data and communicate the data or information based on the data. The electronics subassembly may comprise sensors and associated electronics that, in some embodiments, may perform analysis of sensor data and output results of the analysis and/or recommendations or instructions based on those results. In some embodiments, the electronics subassembly may include a sensor portion comprising one or more magnetic sensors situated to be opposite one or more corresponding magnets on the winder cassette 110, as described more fully below.

The winder cassette 110 cooperates with the strap 115 generally to enable a loose-tensioned but snug fit to the limb, even in the presence of severe swelling. The device 100 does not ordinarily fit tight to the limb, even in the presence of severe swelling. Indeed, the strap 115 and winder cassette 110 are constructed so that the strap 115 may extend and retract as the limb swells and contracts, enabling an essentially constant force to be applied to the limb. In some embodiments, the winder cassette 110 combined with the strap 115 and clasp 120 may be a replaceable component that a user can change out easily if it were to become soiled or broken.

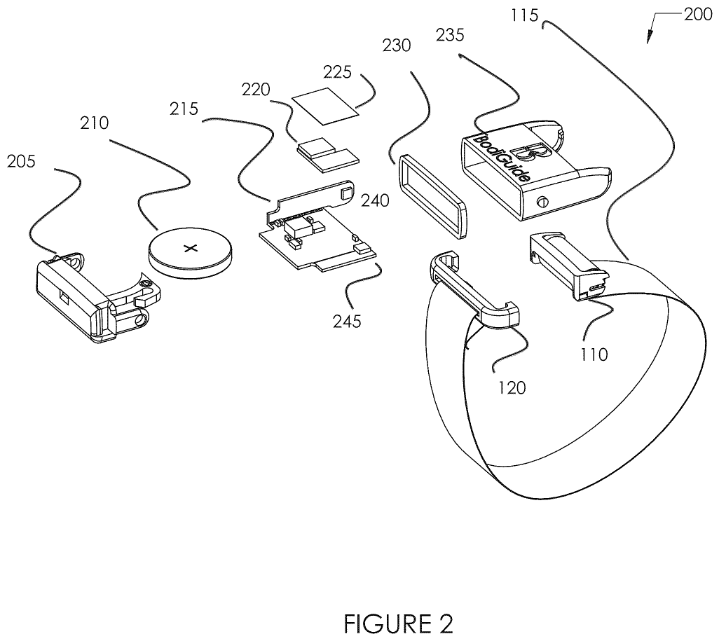

FIG. 2 shows an exploded view of an example measurement assembly 105, winder cassette 110, strap 115, and clasp 120 of a device 200 corresponding to the example illustrated in FIG. 1. An enclosure 235 is configured to house one or more of a battery drawer 205 in which to support a battery 210; an electronics subassembly 215 which includes a radio module 220, an angle magnetic sensor 240, and one or more strap-size magnetic sensor(s) 245; an insulator 225, and a gasket 230. An identification label may be applied to the enclosure 235, although no limitation should be inferred. An etched label, printed label, or the like may be used. Indeed, the device may have no identification label. The measurement assembly 105 may be substantially fluid-tight to prevent fluid ingress.

The battery 210 may be a so-called coin battery of any sort that meets the power and size requirements of the device 200, and in particular the power supplied to components on the electronics subassembly 215 and other components, and of a size that fits within the battery drawer 205. In some examples, the battery 210 may be captured between the electronics subassembly 215 and the top of the enclosure 235. In some embodiments, another portable power source may be used, such as a rechargeable battery, fuel cell, storage capacitor, energy harvested from the patient, energy harvested from the environment, and the like.

The insulator 225 may be an electrical insulator, and so can be positioned to insulate the battery 210 from the electronics subassembly 215 so that the battery 210 can be changed without contacting any components or wiring on the electronics subassembly 215, or the electronics board substrate. In some embodiments, the insulator 225 may be stamped or printed with configuration information, such as version, for the device 200 or any component thereof.

The electronics subassembly 215 may be a printed circuit board and include electronic components that control and carry out sensing of limb circumference. In at least some embodiments, the limb circumference may be used by an on-board control system or transmitted to a remote computing device running an algorithm on data from the sensed limb circumference, outputting messages, and generating graphic results showing measurements over time that can be interpreted by a medical professional (for example) to give insight into the current condition of a patient wearing the device 200. In some embodiments, the electronic components may include one or more magnetic sensors such as magnetic sensors 240, 245, one or more optical sensors, one or more processors, memory, and an accelerometer. The memory may store instructions that, when executed by the one or more processors, cause the one or more processors to carry out various operations described herein. In at least some embodiments, the magnetic sensors may detect limb circumference. In at least some embodiments, the accelerometer may detect patient orientation and motion and output data that may be used by an on-board control system or transmitted to a remote computing device to determine a level of activity of the patient. For example, the device, by information received from the accelerometer, may detect, accumulate, store, and/or transmit accelerometer measurements at a sufficiently high frequency and associate the information with circumference readings to provide an adequate representation of gravity effects and activity on the limb during the time between circumference measurements.

The radio module 220 may transmit a digital or analog signal (e.g., containing patient motion information) to an external device. For example, the signal may be transmitted to a nearby computer, smartphone, tablet, or the like which has processing power to receive the signal, analyze the data provided by the signal, output an instruction or command, and/or relay the signal to a remote computing device such as a server, computer, or database (at a doctor's office or data center, for example).

The gasket 230 is configured and positioned to seal the enclosure 235 and prevent dust or water intrusion that might harm sensitive components inside. For example, the gasket 230 may be positioned between the enclosure 235 and the battery drawer 205. The gasket 230 comprises, without limitation, an insulative material suitable for its purpose.

The measurement assembly 105 may be constructed such that the insulator 225 and electronics subassembly 215, which includes radio module 220, are inserted into enclosure 235. The battery 210 may be joined together on the battery drawer 205 with the gasket 230 positioned where the battery drawer 205 meets the enclosure 235 by a snap fit, to facilitate easy removal of the battery drawer 205 for battery 210 replacement. In this example, the battery drawer 205 may snap into the enclosure 235, compressing the gasket 230 which seals the interior of the enclosure 235 from dust or water intrusion. Then, the winder cassette 110 may be fitted to the measurement assembly 105, by means of detents bringing together the measurement assembly 105 and the winder cassette 110, including the strap 115.

The distal end of the strap 115 is threaded through the body of the clasp 120 and secured by adhering the strap 115 to itself by heat joining with adhesive material the end to the body of the strap 115. But it should be understood that many other methods of joinery such as the strap 115 material bonding to itself or the clasp 120, impinging, and similar means are within the scope of this disclosure.

The clasp 120 can be joined to the electronics subassembly 215 by latch features that engage geometry of the battery drawer 205 in the electronics subassembly 215 in order to encircle the limb.

The winder cassette 110 may be constructed to accommodate different length straps, allowing the device 200 to accommodate a broad range of application from a small wrist to a substantially swollen leg, for example. The sizing of the device may be defined by fixing the length of the strap 115. The winder cassette 110 may be assembled and labeled to identify the strap sizes such as small, medium, large, and extra-large. This sizing data may be collected, stored, and updated as necessary if the “size” is readjusted or calibrated.

In some embodiments, the enclosure 235, battery drawer 205, and/or clasp 120 may be 3D printed by the masked stereolithography (MSLA) process from materials such as Siraya Tech Blu resin and Siraya Tech Blu mecha nylon resin. The gasket 230 may comprise Poron (polyurethane foam). The strap 115 is generally flexible and inelastic, and may be made of a biocompatible, porous material for patient comfort. As one example, the material can be an open weave, 80 threads per inch polyester with fused edges, 0.008″ Teslin (PPG, Barberton, OH) or Tyvek (Wilmington, DL) or a nonwoven open fabric such as embroidery stabilizer.

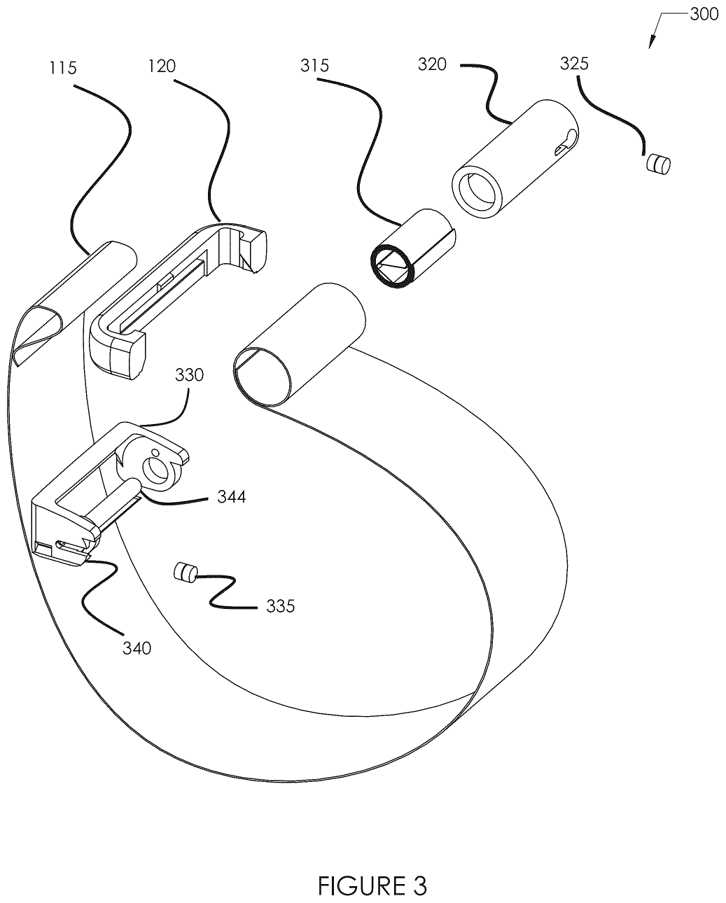

FIG. 3 shows an exploded view of the winder cassette 110, strap 115 and clasp 120 that remains when the measurement assembly 105 is removed in some embodiments. The winder cassette 110 may comprise a winder cassette frame 330, a spring 315, and a spool 320. The winder cassette frame 330 may include a capture feature 340 which may include a strap-size magnet 335. The capture feature 340 may further include a spring coupler formed by a pin 344 joined to the winder cassette frame 330. The spool 320, which may be cylindrical, includes an angle magnet 325. The strap 115 may wrap around spool 320 when installed in the winder cassette frame 330 as described below. In some embodiments, the winder cassette 110, strap 115, and/or clasp 120, individually or as a combination that includes any two or all three, may be replaceable.

The winder cassette 110 may be constructed such that the spring 315 is attached to the capture feature 340 of the winder cassette frame 330 and the inside of spool 320. The spring 315 may provide a substantially constant spring force load (for example, approximately 10-40 grams) sufficient to snug the strap 115 to the limb without unnecessary, uncomfortable pressure.

The strap 115 is attached and wound around the spool 320. The angle magnet 325 may be pressed or otherwise fitted into an opening, gap, or recess of the spool 320 such that it opposes the angle magnetic sensor 240 on the electronics subassembly 215 of the measurement assembly 105. The strap-size magnet 335 may be pressed or otherwise fitted into an opening, gap, or recess of the winder cassette frame 330 such that it opposes one or more of the strap-size magnetic sensor(s) 245 on the electronics subassembly 215 of the measurement assembly 105. The magnetic sensors 240 and 245 are configured and positioned to sense the magnetic fields of the magnets 325 and 335 through the enclosure 235.

The action of increasing and decreasing the length of the strap 115 as the limb expands and contracts may be accomplished with a tensioning mechanism comprising the winder cassette frame 330 and spring 315 within the spool 320 that may house a portion of the length of the strap 115 that is wrapped around the spool 320. In some embodiments, the spring 315 may be a constant tension spring. As the limb expands, the strap 115 unrolls, increasing the length of the strap 115 to accommodate the increased circumference of the limb while maintaining a constant tension of the strap 115 around the limb by a constant force applied by the spring 315. The tension of the strap 115 may be matched to the interstitial fluid pressure and elasticity of the skin such that the device expands and contracts without creating more than a negligible indentation in the limb. In this regard, and in conjunction with the other embodiments described herein, it is understood that constancy of the force and tension need not be exact but are within a reasonable tolerance that enables the device to perform its function of measuring limb circumference, and particularly differences thereof relative to a baseline or other reference, in accordance with the principles outlined in this disclosure.

The angle magnet 325 fitted in spool 320 may be sensed by the angle magnetic sensor 240 such that as the rolling and unrolling of the strap 115 causes the spool 320 to rotate, the degree of rotation of the spool 320 may be recognized by a signal output by the angle magnetic sensor 240 and received at the electronics assembly 215, which is electrically connected to the angle magnetic sensor 240.

The angle magnet sensor 240 may be made up of a plurality of resistive elements that are arranged to output a variable set of signal strength voltages. In at least one embodiment, these correspond to the sine and cosine of the degree of rotation of the angle magnet 325 embedded in the spool 320 in relation to the angle magnetic sensor 240. This can be accomplished by a full bridge sensor, for example, with an underlying spintronic technology (NVE corporation, Eden Prairie, MN produces suitable sensors at this time) as this produces a very low power construction that is relatively insensitive to distances and axial misalignment between the angle magnet 325 and the angle magnet sensor 240. As the diameter of the spool 320 defines a known circumference by the formula C=Pi×D, the diameter can be converted to precise changes in length measurements as the strap 115 is expanded and contracted. The diameter of the spool 320 may be determined/calibrated in manufacturing for each device.

In addition to measuring the degree of rotation of magnet 325, the circuit may retain information about rotation history. In this example, quadrature detection may be implemented in software or hardware to track the current revolution or number of revolutions. In at least one embodiment, quadrature detection can be accomplished by use of a comparator circuit that is connected to interrupt functionality on the processor. But in other implementations dedicated circuit counting and logic components or integrated functionality within the processor itself may serve this function. As the strap 115 wraps around the spool 320 for greater than 360 degrees, a count of rotations is maintained. The count of rotations in conjunction with the current angle measurement enables the software to calculate the total number of degrees of rotation. The diameter of the spool 320 coupled with the total number of degrees of rotation in conjunction with the manufacturing strap 115 length in the unexercised spring 315 condition, the length of the measurement assembly 105, and clasp 120 may determine the total circumference measurement.

There may be an additional consideration to calculating total length. As suggested above, the strap 115 as it wraps around the spool 320 changes the effective diameter of the wrap and the circumference around the spool 320 as layers stack on top of one another. To account for this condition it is possible to recognize the rotation history and manufacturing design of the winder cassette 110 and apply a change in the diameter consistent with the thickness of the strap 115 and the number of wraps of strap material about the spool 320.

The length of the strap 115 when the spring 315 is in the relaxed and unexercised condition is controlled when the winder cassette 110 is manufactured. If the angle of rotation of magnet 325 is consistent with a reference that corresponds to the manufactured relaxed orientation and the rotation history shows no additional winding, and there is no dithering of the angle from being worn, it may be assumed that the device is not being worn. In some embodiments, the spring 315 may retract the strap 115 to the home location, which can be read by the software that is executed to interpret the signals representing the detected angle of spool 320, with an indicator or message output. The detection of these conditions also can be used to indicate whether a device is being worn. The data from the device 100 in the “not worn” condition may be ignored in some embodiments in evaluating the condition of the wearer. Further, if this condition persists, the patient may be contacted or examined regarding any issues with wearing the device 100.

The device 100 may accommodate a range of limb size in at least two ways. For instance, constructing the winder cassette 110 as in the example explained above, substantial spooling of the strap 115 may be achieved. In this example, the winder cassette 110 may accommodate, e.g., 100 millimeters of strap travel for circumference change. Additionally, or alternatively, additional range may be achieved by changing the total length of the strap to create winder cassettes 110 of various sizes such as small, medium, large, and extra-large. One benefit of this sizing design is that the winder cassette 110 can allow for substantial variations in patient limbs sizes and further substantial variation in ankle circumference due to the presence or absence of edema. This may reduce the difficulty of sizing and fitting of the device to a particular patient and can accommodate large circumference limbs experienced by patients with lymphedema. This reduces or eliminates the need for custom-sizing the strap 115 to a particular individual and simplifies the size fitting procedure.

This construction may achieve at least two types of measurement. The first is a relative measurement quantifying only the changes in circumference associated with the change in spool from its home position as a result of the expansion and contraction of the limb The second type of measurement is an absolute measurement that can determine the total circumference of the limb by combining the relative measurement, length of the electronic assembly 105, clasp 120, and unexercised length of the strap 115.

In the current example, the strap-size magnet 335 in the winder cassette 110 may communicate with one or more strap-size magnetic sensor(s) 245 in the electronics subassembly 215. Several strap-size magnetic sensors 245 may be positioned so as to recognize a plurality of strap sizes by the relative position and orientation of the magnetic field generated by the strap-size magnet 335. In at least one embodiment, a single strap-size magnet 335 may be used to recognize as many as four strap sizes.

The strap-size magnetic sensor(s) 245 can recognize the presence and/or the orientation of the strap-size magnet 335 in the winder cassette 110. In some embodiments, two strap-size magnetic sensors 245, such as Hall effect sensors, can each output a signal in the presence of a north pole field or a different signal in the presence of a south pole field. By the arrangement of the relative position of the strap-size magnetic sensors 245 and the orientation of the strap-size magnet 335 in the winder cassette 110, it is possible to detect and communicate five orientation conditions. For example, when no magnetic field is sensed, the winder cassette 110 is determined (e.g., decoded) as not present; when the magnetic field axis is perpendicular to the circuit board supporting the strap-size magnetic sensors 245, a north-north or south-south condition may be sensed; and when the magnetic field axis is parallel to the axis along the strap-size magnetic sensors 245, a north-south or a south-north condition may be sensed. Sensing the presence or absence of the winder cassette 110 with the strap-size magnet 335+strap-size magnetic sensor 245 coupling may allow for the recognition of a removal and replacement event. In addition, sensing the orientation of the strap-size magnet 335 facilitates recognition of a size of the winder cassette 110 such as small, medium, large, and extra-large. Winder cassettes 110 of different strap 115 lengths may be built with the strap-size magnet 335 oriented such that the field presented to the strap-size magnetic sensor(s) 245 in either intensity or field orientation can be read by the circuit and interpreted to communicate the size of the winder cassette 110 attached to the measurement assembly 105.

In some embodiments, the winder cassette frame 330 and spool 320 are 3D printed by the masked stereolithography (MSLA) process from materials such as Siraya Tech Blu resin and Siraya Tech Blu mecha nylon resin. The spring 315 may be 125 millimeters long by 10 millimeters width by 0.025 millimeters (0.001 inch) full hard stainless steel shim stock (Precision Brands, Downers Grove, IL). The angle magnetic sensor 240 may be a giant magneto resistor angle sensor such as AAT101-10E Full Bridge Angle Sensor by NVE (Eden Prairie, MN). The strap-size magnetic sensor(s) 245 may be dual output unipolar hall effect switches such as AH1389 by Diodes Inc (Plano, TX). The magnets 325 and 335 are neodymium available from various suppliers.

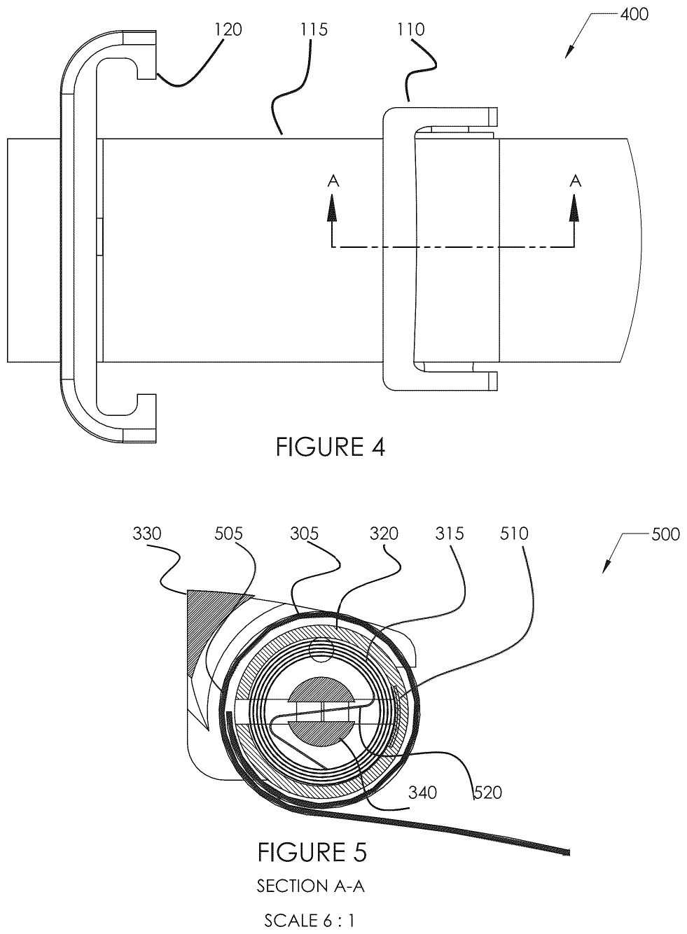

FIG. 4 is a top view of the winder cassette 110 relative to the strap 115 and clasp 120. From this view 400 a partial cross section A-A of the winder cassette 110 is located.

FIG. 5 is the partial cross section view A-A in FIG. 4 of the winder cassette 110 in the assembled condition.

In some examples, the capture feature 340 may comprise a pin 344 fixed within the winder cassette frame 330, such as within one or more tabs, grooves, or holes in the wire cassette frame walls to receive corresponding ends of the pin, or extensions from one or both walls on which to mount hollow end portions of the pin, for example. In some embodiments, one or both ends of the pin 344 may be extruded from or fixed to the wall or walls. In such embodiments, the spring 315 may be attached to the capture feature 340 that mounts to the winder cassette frame 330 by, e.g., inserting one end of the spring 315 into a gap in the pin 344 of the capture feature 340 and wrapping or bending the spring 315 around the pin.

The coil spring 315 may have a narrow wire or broad band-like construction, for example. To aid with fixing the spring 315 to the capture feature 340 of the winder cassette frame 330, the end of the spring 315 that is inserted into the gap may be wrapped or bent to wrap at least partially around the pin 344. A bend configuration 520 comprised of two bends in the spring 315 may engage the capture feature 340 as shown in FIG. 5. The distal end of the spring 315 may be joined by adhesive 510 to the spool 320. The spool 320 may in turn be attached to the strap 115 by an adhesive 505. No limitation on a particular type of fixing or adhesive should be inferred. This communicates the spring 315 force from the capture feature 340 in the winder cassette frame 330 by the bend configuration 520 of the spring 315. The attachment of the winder cassette frame 330 to the spring 315, adhered 510 to the spool 320, and in turn adhered 505 to the strap 115, tensions the assembly. The action of rotating the angle magnet 325 opposite the angle magnetic sensor 240 as the limb expands and contracts may be accomplished with this tensioning mechanism.

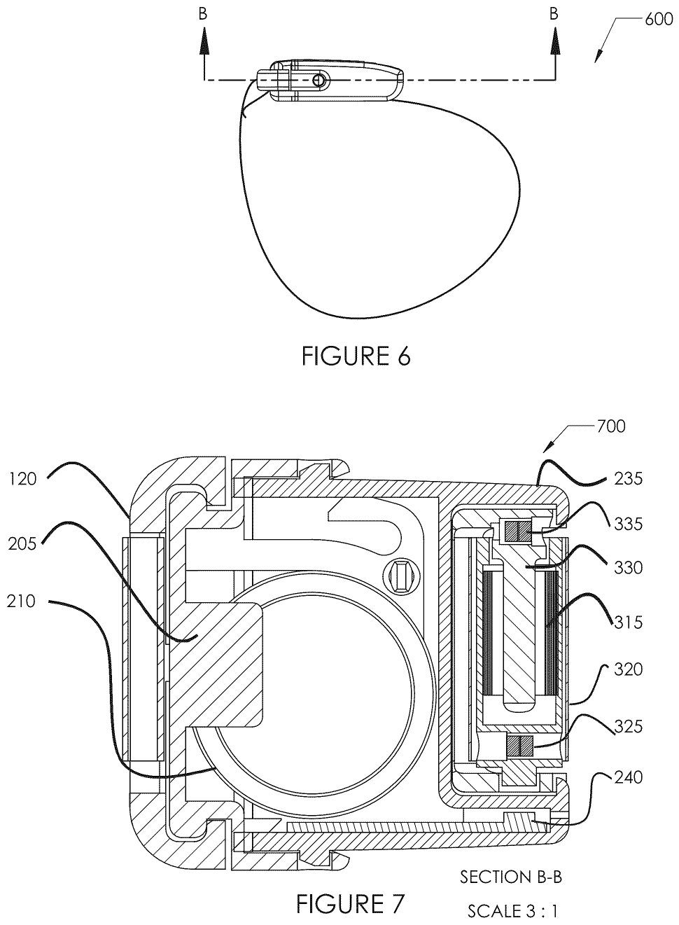

FIG. 6 is a side view 600 of the device, which may correspond to the device 100. In practice, the device 100 may drift to its operative, repeatable home location on the limb by the influence of gravity and patient movement, as influenced by friction between the strap 115 and the limb.

FIG. 7 is a detail section view 700 through the measurement assembly 105, winder cassette 110, strap 115 and clasp 120 of the example device illustrated in FIG. 6, taken along line B-B. This illustrates the engaged use position of the clasp 120 to the battery drawer 205 and the position of the battery 210 in the enclosure 235. A scale of 3:1 is indicated to give a sense of the size of the device, which may correspond to the device 100. However, the scale will change depending on the size of the figure as presented on the page. That is, zooming in or out will affect the scale, and thus 3:1 should not be considered limiting.

On the right side of FIG. 7, the winder cassette 110 is in its use position. The winder cassette frame 330, spool 320, spring 315, angle magnet 325 and strap-size magnet 335 work in conjunction to determine the degree of rotation of the spool 320, the presence of the winder cassette 110 and the overall length of circumference measurement about the limb of the wearer, determined as described elsewhere herein.

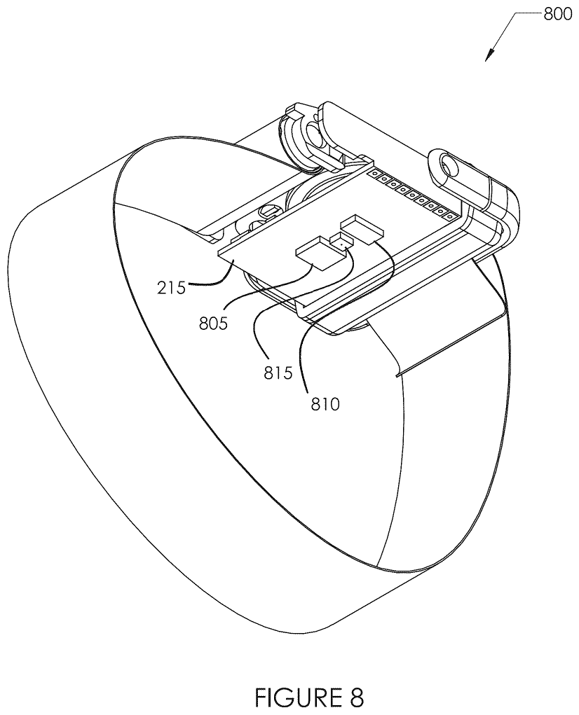

FIG. 8 is an isometric view of the device, which may correspond to the device illustrated in FIG. 1 with the enclosure of the measurement assembly removed. The view orientation shows the electronics that are in close proximity to the limb with the rear enclosure 235 removed. On the electronics subassembly 215 there may be components for the sensing of heart rate, SPO2, NIBP and like physiological parameters. In the example shown an optical emitter 815 that may emit light in the green, red and infrared wavelengths is placed centrally and in close proximity to the wearer. Light from this emitter component travels into the skin of the wearer. Some of this light is reflected back and is sensed by detector components 805 and 810. The returned light is sensed and evaluated by the software. The raw or evaluated measurements can then be transmitted to other computing devices.

In some embodiments, the device 100 may use orientation information detected by the accelerometer to detect when the patient is in the horizontal position or resting. Resting heart rate is sampled. Variation in resting heart rate is known to change as a patient retains fluid. These heart rate readings can be used to increase confidence in the interpretation of circumference measurements.

Limb circumference measurements and limb orientation data may be taken continuously at a regular interval and processed to produce a personal Daily Swelling Pattern for the subject wearing the device. The Daily Swelling Pattern is characterized by a minimum limb circumference that occurs when the subject is lying down and at a maximum circumference after the subject has been in a vertical position such as standing or sitting for a period of time specific to that individual.

Trends in the fluid gain or loss can be computed for specified time periods such as days, weeks, or months. Fluid gain/loss and fluid gain/loss trends can be compared to threshold values to identify conditions of interest. The system takes actions specific to the condition of interest including sending messages and alerts to the user as well as support personnel such as family caregivers, chronic care management, and/or clinical personnel.

The rate at which fluid redistributes itself in the body on rising to a vertical orientation may be indicative of the viscosity of the interstitial fluid; changes in viscosity are known to be related to heart failure decompensation due to changes in protein levels in the interstitial fluid. This is typically evaluated by a physician pushing a finger firmly against the ankle of the patient and seeing if the “dent” produced rebounds quickly. If the dent is slow to rebound, the condition is described as pitting edema. This is a significant medical sign and a useful part of the diagnostic method in characterizing the condition of the patient.

The disclosed techniques can characterize the rate of change of the redistribution of interstitial fluid. By taking a plurality of measurements when the patient moves from a supine to upright orientation, typically in the morning, it is possible to track the time it takes for the redistribution of interstitial fluid associated with the change in direction of the gravitational force. This rate of change measurement is directly related to the viscosity of the interstitial fluid. Being able to recognize interstitial fluid viscosity and changes associated with it can further inform the medical practitioner or computed algorithm about changes in the disease state of the patient, as fluid viscosity offers insight to the underlying cause of fluid loading (e.g., change in protein levels in the interstitial fluid).

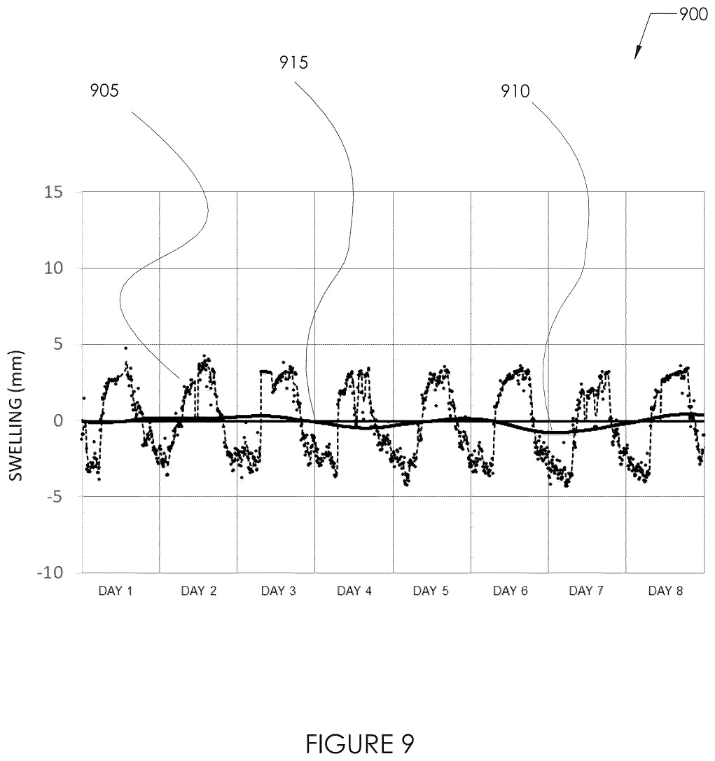

FIG. 9 shows a plot 900 of normal swelling (e.g., swelling of a limb) over a time period of 12 days for a hypothetical heart failure patient. The reference number 905 is associated with a plot of the circumference readings captured by the device (e.g., device 100) vs. time and shows the repetitive Daily Swelling Pattern over the 12 days. The reference number 910 is associated with the rolling average of the ankle circumference. In some embodiments, an exponential moving average may be used. The reference number 915 is associated with a user's normal baseline circumference that is derived from circumference readings measured during a user's normal or “dry” state in the example of the heart failure patient.

A swelling pattern can be represented by an “average” circumference considering the minimum and maximum circumferences associated with the swelling pattern.

The Baseline Swelling Pattern or the Baseline Average Swelling is identified as the Daily Swelling Pattern or Average Daily Swelling detected when the subject is in a normal state of health usually referred to as a “dry” state for heart failure patients. The systems can compute or adjust the normal baseline over a period of use. The system maintains the normal baseline for comparisons to compute fluid gain/loss.

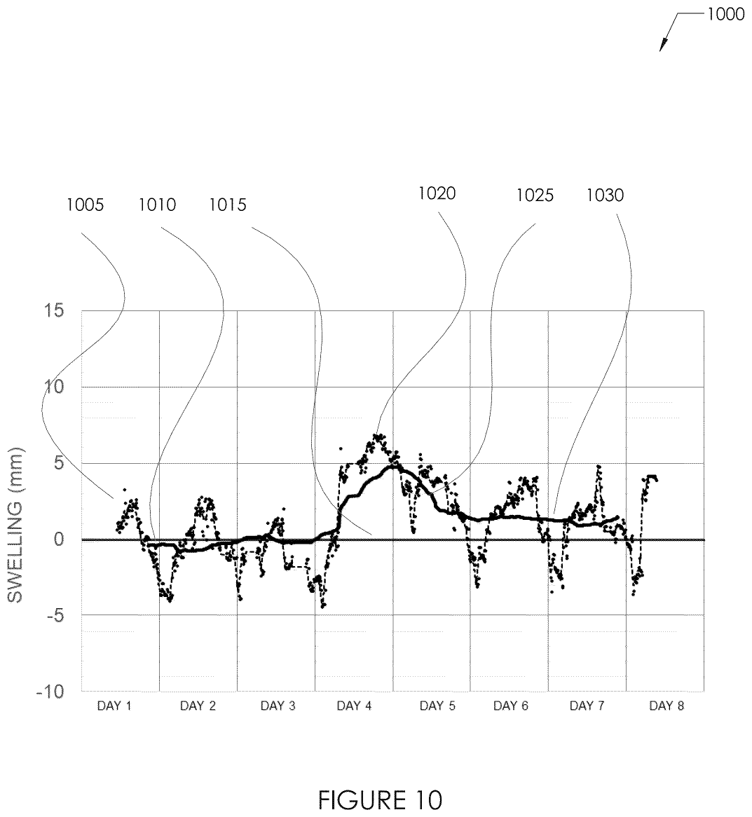

FIG. 10 shows an 8-day plot 1000 of a compensated swelling event (e.g., swelling of an ankle) associated with fluid retention related to increased salt intake for a hypothetical heart failure patient. The reference number 1005 is associated with a plot of the circumference readings captured by the device (e.g., device 100) vs. time. The reference number 1010 is associated with the rolling average of the ankle circumference. A similar plot can be made if using an exponential moving average. The reference number 1015 is associated with the patient's normal baseline circumference that is derived from circumference readings, e.g., circumference readings measured during a user's normal or “dry” state in the case of the heart failure patient. The reference number 1020 shows the deviation in the Daily Swelling Pattern that may be associated with fluid retention for this patient, in this case intake of high-salt meals for two consecutive days. The reference number 1025 points to the corresponding change in average circumference that occurs during the swelling event. The reference number 1030 points to the return to normal swelling in the days following the event as the patient's body compensates and eliminates the extra fluid.

In FIG. 10 there are quite different waveform plots of individual measurements 1005. These patterns are different between individuals and days; this individual's pattern is substantially less regular than the previous example. But, the moving average trend line 1025 indicates a 4-millimeter increase in swelling occurring over a 2 day period. This coincided with a heavily salty meal. The swelling subsided 1030 over time as the body compensated for this infusion.

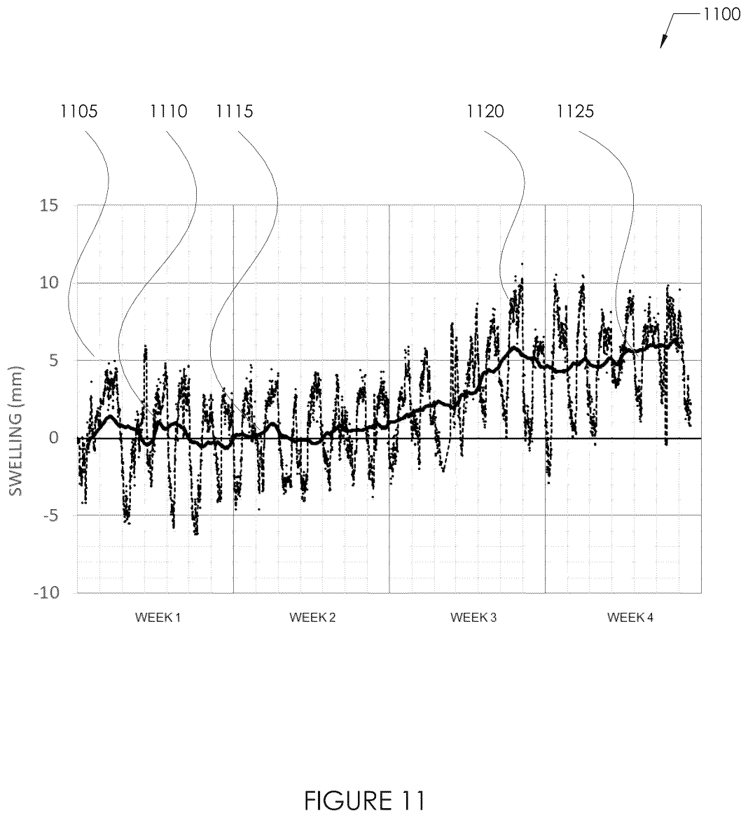

FIG. 11 shows a plot 1100 of a hypothetical decompensated fluid retention episode such as one preceding a heart failure hospitalization. The reference number 1105 is associated with a plot of the circumference readings captured by the device (e.g., device 100) vs. time over four weeks. The reference number 1110 is associated with the rolling average of the ankle circumference. A similar plot can be made using an exponential moving average. The reference number 1115 is associated with the patient's normal baseline circumference that is derived from circumference readings measured during a user's normal or “dry” state in the case of heart failure patients. The reference number 1120 shows the deviation in Daily Swelling Pattern that may be associated with fluid retention, in this case a trend associated with decompensation related to a condition that requires clinical intervention. The reference number 1125 is the corresponding change in average circumference associated with increasing fluid retention.

The plot 1100 of FIG. 11 shows yet a different pattern of measurements. The excursions in circumference measurement can exceed 10 millimeters over a day while the Average Daily Swelling signal change of several millimeters is the signal of interest. The potential error, deviating from the arithmetically processed measurements, is larger than the signal for a single measurement. This patient had a much larger daily range of measurements 1105 as well as a substantial increase in monthly excursions. On essentially the same location on the same ankle, there is a swelling range of 17 millimeters over the month. Of interest is that, during the first two-week period, the average swelling remains in a plus or minus 1 millimeter range 1110. However, during the subsequent two-week period, the Average Daily Swelling increases to almost 6 millimeters 1125 above the nominal baseline 1115. This represents a substantial and persistent trending of the circumference data indicative of increases in patient fluid volume.

This persistent trending can be compared with the individual patient's history as well as patients who might have similar characteristics such as, but not limited to, age, height, weight, left ventricle ejection fraction, comorbidities, and/or similar measures. Additionally, other physiological measures such as, but not limited to, heart rate, SpO2, NIBP, temperature, footfall impact, pace, arrythmia, tachycardia, bradycardia, atrial fibrillation, and/or heart rate variability may also be considered in conjunction with these measurements. Processing this information, using appropriate correlative algorithms, may enable a real-time, consistent, continuous, and/or instant acute evaluation of the trending event severity and predict the likelihood of an impending decompensation event. In some embodiments, trained machine learning models or rules application algorithms may be utilized and updated in accordance with feedback (human or machine) from the patient's experience and/or from a population of patients to constantly improve the accuracy of these predictions; the more measurements taken; the more accurate the predictions, especially in order to minimize interpolation errors and determine an accurate model of changes of daily swelling of a limb. When a substantial swelling event exceeding a predetermined threshold stored on the device or remotely is detected, a patient or their caregiver may be notified to take actions to change overall patient activity, contact their medical provider, change the amount of a treatment such as a diuretic, add or subtract other medications, or implement other methods that may alter the course of the disease. In some examples, the responsive actions are urgently provided; in others, such as diet change, the responsive actions may be suggestions or instructions. Changes in medication dosage would normally follow a predefined treatment plan from the patient's physician, where an additional diuretic dose might be indicated when the patient has a substantial swelling event, for example.

Given the small size of the arithmetically processed signal of interest compared to the range of circumference measurements over a day, it is desirable to minimize sampling rate induced distortions of the underlying, continuous changing, physiology. Linear interpolations that truncate the actual limb swelling excursion values can materially change the value of the calculated signal of interest. In the simplest of examples, a plot of circumference measurement at a random time during a week could yield substantially different results that might substantively contradict a more densely sampled identical patient and condition. This is particularly true when evaluating the rate of change of circumference associated with changes in gravitational orientation of the patient.

In the nonlimiting examples shown in FIGS. 9, 10, and 11, measurements are taken every 10 minutes. But it is understood that different intervals are within the scope of this disclosure.

These individual measurements may be mathematically aggregated over a period of time, such as a day, 48 hours, or any time suited to the monitoring and analysis. In this example it is by means of a rolling average; an exponential moving average is also contemplated. A series of these aggregated measurements are then compared for evidence of deviation or divergence. In the example of FIG. 9, for example, there are a series of points representing individual circumference measurements. It should be noted that there is a fair amount of variability in these measurements.

Measurements for this individual ranged 10 millimeters for the same position on the same ankle depending on the day and time. But the aggregate daily oscillation in measurements is in fact quite stable. The range for this individual was about a millimeter as can be seen in the average daily circumference trend line 915.

This paper describes a method of capturing a plurality of measurements of minimum limb circumference typical of a narrowing of the limb at an ankle or wrist, for example, it should be noted that the teachings herein are applicable whether the minimum limb circumference is at a narrow or narrowing point or at a location on a constant circumference, such as a constant cylinder. And limb circumference is a close proximate of interstitial fluid volume. And interstitial fluid volume is a key medical concern in patients with conditions such as heart failure decompensation, kidney function and similar physiological conditions. Increases in interstitial fluid volume are associated with heart failure decompensation. It should be clear from FIGS. 9, 10 and 11 that a single measurement, perhaps at a doctor's appointment, would yield dramatically different and contradictory results depending on the random time of day of the appointment. This has limited doctors to gross approximation of the presence or absence of swelling, such as pressing their finger into your ankle. Lacking practical and rigorous medical measurement has constrained the usefulness of this physiological parameter. It is with the careful quantification and tracking over time that changes in fluid volume, that are pertinent physiological information, are apparent. Average changes of a few millimeters can present as a significant sign of disease progression but are invisible at a single inspection, or daily inspection, and require more precise measurement than is possible by visual inspection alone.

It should be noted that a single measurement at a fixed time of day would not reliably yield accurate results. As can be seen in FIGS. 9, 10 and 11, extreme excursions of the measurement do not necessarily occur at the same time each day.

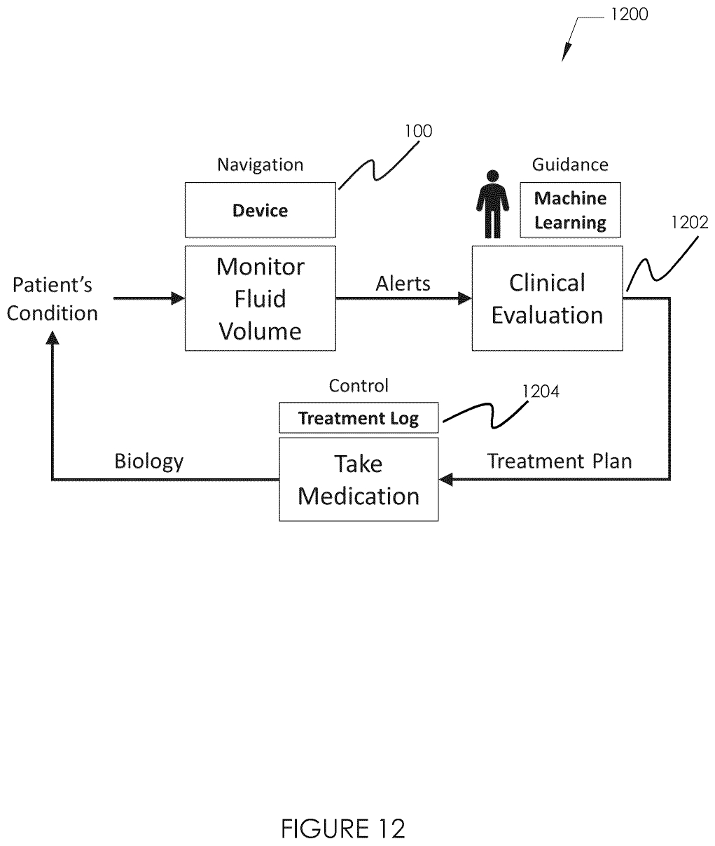

FIG. 12 shows an example of a loop 1200 that illustrates the efficacy of integrating interstitial fluid volume monitoring via the device 100 with a determined outcome that may automatically follow according to techniques and concepts described herein. That is, the absolute measurements by the device 100 and/or trends in those measurements over time (navigation) may be output as alerts (e.g., discrete data, waveforms, and/or simple notifications) for clinical evaluation 1202 by a human or by artificial intelligence based, e.g., on machine learning principles (guidance). Results of the clinical evaluation 1202 may be output as a treatment plan (which may include, among other things, advice or instructions to the patient, a prescription for medicine or therapy, and/or the like) and optionally entered into a treatment log 1204 (control). The loop 1200 may close with analysis of the patient's response to the treatment plan, completing a navigation-guidance-control integration of patient support made possible by the device 100 and its operations/functions to monitor and/or measure swelling, and make from them not simple data for evaluation but real-time monitoring-to-action, obviating the need for many consultations or interventions often previously considered a best, even necessary, practice.

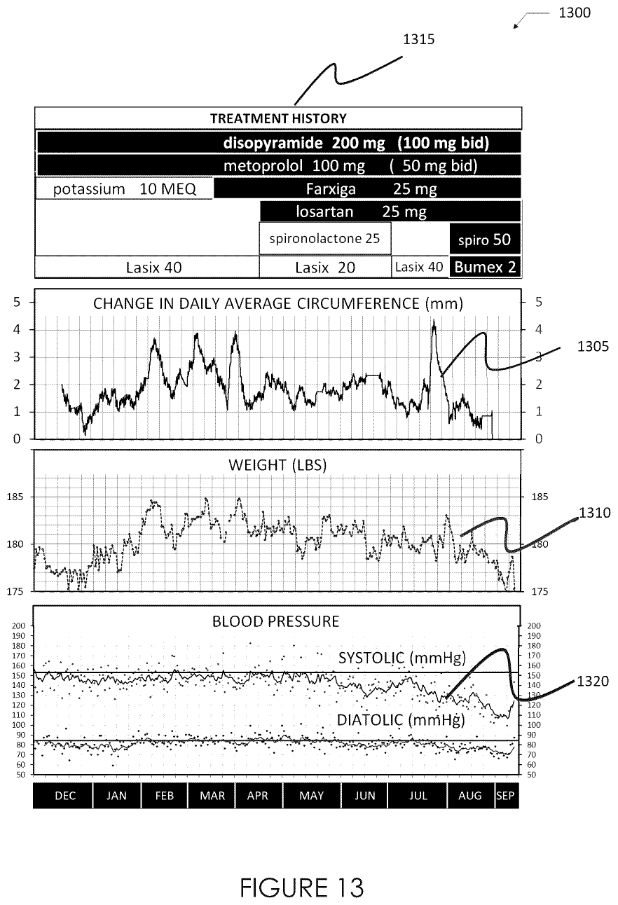

The plot 1300 of FIG. 13 illustrates an example of an integrated medication treatment log and anklet circumference history. The plot allows the medical practitioner to determine an outcome associated with a prescribed treatment plan. The circumference information combined with the prescribed treatment plan may be used to determine whether the medication achieved the desired outcome and adjust the medication as necessary to achieve the desired result. The Average Circumference 1305 is the patient response to the medical Treatment Plan 1315. The physician evaluates the response to determine effectiveness and make adjustments to the Treatment Plan 1315 to achieve the desired result. The non-invasive blood pressure profile 1320 may be used to assess the patient's ability to tolerate medication adjustment that may reduce fluid retention while staying within the allowable blood pressure range.

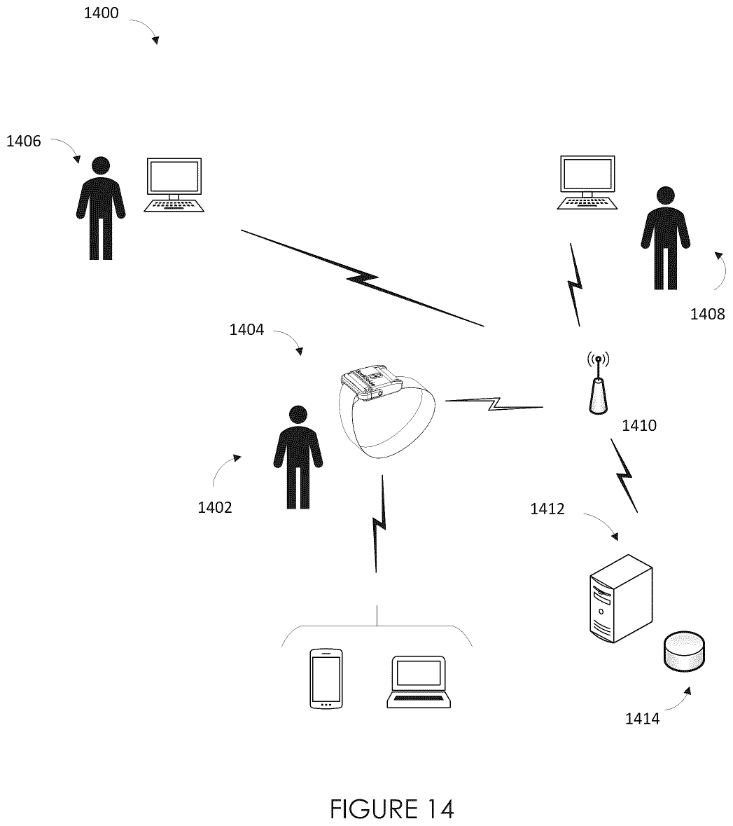

FIG. 14 illustrates an example architecture 1400 to implement monitoring changes in volume and properties of human interstitial fluid in a patient 1402. The illustrated architecture 1400 includes a device 1404, a health care entity 1406, a personal contact 1408, and a wireless access point 1410, although this is an example only and other configurations including more components or fewer are contemplated.

The health care entity 1406 may include, without limitation, a medical facility, caregiver, and/or other personnel associated with patient care. A caregiver or other personnel may operate one or more computing devices as part of their care function.

The personal contact 1408 may include support personnel operating one or more computing devices connected to a database server 1412 via a wired or wireless communication link (e.g., the Internet or other wireless and/or wired connection) using SMS messages, WIFI protocols, Bluetooth protocols, and the like. The personal contact 1408 may also include a personal representative for the patient, family member, or other individual set up to receive information derived from the device 100.

A control system may include the database server 1412 connected to a database 1414. The database 1414 may store pertinent data about the patient 1402 (such as patient history, a patient record, and the like), trigger event levels, and addresses to which messages (e.g., notifications, alerts, and the like) are to be sent.

Information from the device 1404 can travel several alternate paths depending upon the implementation details. For example, the information may be input into a computing device (e.g., a patient desktop computer, a patient cellular telephone, a patient portable computer, and the like), connected to the database server 1412 (or a web server) via the Internet, cellular gateway, or other network. The computing device may transfer the feedback information to the database server 1412 directly or via the access point 1410. The device 1404 may communicate the device messages to the computing device for transmission thereby to the database server 1412.

The device 1404 may continuously measure and store position measurements on the device itself or remotely, e.g., at the database 1414, at a predefined frequency. The positions may be interpreted as a relative circumference measurement when compared to an arbitrary reference or an absolute circumference measurement when combined with the size information which establishes a relationship between a position of the spool 320 and a known circumference.

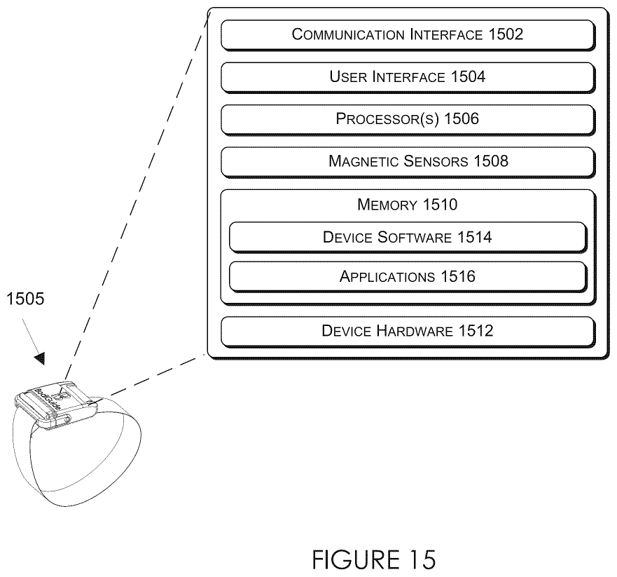

FIG. 15 illustrates various components of a measurement assembly 1505 arranged in accordance with one or more embodiments described herein. The measurement assembly 1505 may correspond to the measurement assembly 105 shown in FIG. 1, for example. In the illustrated example, the measurement assembly 1505 may be configured to measure the circumference of a limb using one or more sensors, such as magnetic sensors, by detecting the extension or retraction of a strap 115 attached to a winder cassette 110 coupled to the measurement assembly 1505.

As illustrated in FIG. 15, the measurement assembly 1505 may include one or more of a communication interface 1502, a user interface 1504, one or more processors 1506, one or more magnetic sensors 1508, memory 1510, and device hardware 1512.

The communication interface 1502 may include wireless and/or wired communication components that enable the measurement assembly 1505 to transmit data to and receive data from other networked devices via a communication network such as that described with respect to FIG. 14.

The user interface 1504 may enable a user to provide input and receive output from the measurement assembly 1505, including for example providing one or more input to initiate device activation and/or set metadata, tags, communication parameters, monitoring parameters, etc. The user interface 1504 may include a data output device (e.g., visual display, audio speakers), and one or more data input devices. The data input devices may include, but are not limited to, combinations of one or more of touch screens, physical buttons, cameras, fingerprint readers, keypads, keyboards, mouse devices, microphones, speech recognition packages, and any other suitable devices or other electronic/software selection methods.

The processor(s) 1506 and the memory 1510 may implement an operating system. The operating system may include components that enable the measurement assembly 1505 to receive and transmit data via various interfaces (e.g., the user interface 1504, the communication interface 1502, and/or memory input/output devices), as well as process data using the processor(s) 1506 to generate output. The operating system may include a display component that presents output (e.g., displays data on an electronic display, store the data in memory, transmit the data to another electronic device, etc.). Additionally, the operating system may include other components that perform various additional functions generally associated with an operating system.

The magnetic sensors 1508 may be configured and located within the enclosure of a wearable device of the type described herein, working in conjunction with electromagnetic circuitry to detect the amount of rotation of a magnet associated with spooling and unspooling of the strap 115 which corresponding with a circumference of the limb.

The memory 1510 may be implemented using computer-readable media, such as computer storage media. Computer-readable media includes, at least, two types of computer-readable media, namely computer storage media and communications media. Computer storage media includes volatile and non-volatile, removable and non-removable media implemented in any method or technology for storage of information such as computer-readable instructions, data structures, program modules, or other data. Computer storage media includes, but is not limited to, Random-Access Memory (RAM), Dynamic Random-Access Memory (DRAM), Read-Only Memory (ROM), Electrically Erasable Programmable Read-Only Memory (EEPROM), flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device. Computer readable storage media do not consist of, and are not formed exclusively by, modulated data signals, such as a carrier wave. In contrast, communication media may embody computer-readable instructions, data structures, program modules, or other data in a modulated data signal, such as a carrier wave, or other transmission mechanism.

The memory 1510 may include the operating system, device software 1514, and one or more applications 1516, as well as data gathered by the magnetic sensors 1508 or input by a user or received from a remote source. The application(s) 1516 may include any application software executable by the one or more processors 1506, including but not limited to applications that facilitate the functions of the wearable device 1505 for detecting extension and retraction of the strap 115 by detecting and processing the magnetic field of a magnet within the spool 320 and data derived therefrom; manipulation, formatting, addition, deletion, or modification of metadata; and image or data processing, for example.

The device software 1516 may include software components that enable the measurement assembly 1505 to perform functions. For example, the device software 1516 may include a basic input/output system (BIOS), Boot ROM, or bootloader that boots up the measurement assembly 1505 and executes the operating system following power up of the measurement assembly 1505.

The device software 1516 may include software components that compute a relative time associated with the data collection that can be resolved to an actual time within other computer systems that are synchronized with a universal time reference.

The device hardware 1512 may include additional hardware that facilitates performance of the user interface 1504, data display, data communication, data storage, and/or other device functions.

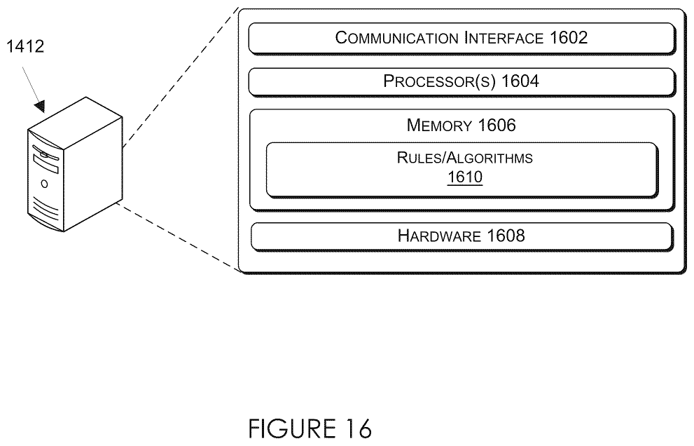

FIG. 16 illustrates an example of the database server 1412 shown in FIG. 14. The database server 1412 may include a communication interface 1602, one or more processors 1604, memory 1606, and hardware 1608. The communication interface 1602, like the communication interface 1502 of the measurement assembly 1505, may include wireless and/or wired communication components that enable the database server 1412 to transmit data to and receive data from the measurement assembly 1505 and other networked devices via a communication network such as that described with respect to FIG. 14.

The processor(s) 1604 and the memory 1606 may implement an operating system. The operating system may include components that enable the database server 1412 to receive and transmit data via various interfaces (e.g., the communication interface 1602 and/or memory input/output devices), as well as process data using the processor(s) 1604 to generate output. The operating system may include a display component that presents output (e.g., displays data on an electronic display, store the data in memory, transmit the data to another electronic device, etc.). Additionally, the operating system may include other components that perform various additional functions generally associated with an operating system.

The memory 1606 may include the operating system, device software, and one or more applications, as well as data gathered by the magnetic sensors 1508 or input by a user or received from a remote source. The applications may include any application software executable by the one or more processors 1604, including but not limited to applications that train and/or execute rules or machine learning models and algorithms 1610 to process data received from the measurement assembly 1505, including generating waveforms of circumference changes, interpreting data, applying activity information, subject medical history, medication treatment plans, and other data, generating predictive output for analysis and/or feeding back to update or retrain models, and the like.

The hardware 1608 may include additional hardware that facilitates performance of data display, data communication, data storage, and/or other device functions.

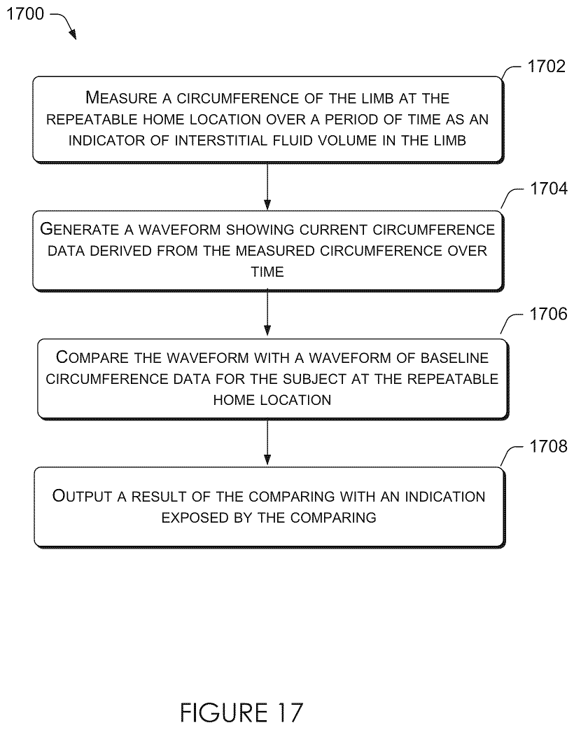

FIG. 17 is a flow diagram of an example process 1700 that may be performed at least in part by the measurement assembly 105 for measuring limb circumference of a subject, generating a waveform of the measurements over time, and outputting indication of a condition exposed by the waveform. The process 1700 is illustrated as a collection of blocks in a logical flow chart, which represents a sequence of operations that can be implemented in hardware, software, or a combination thereof. In the context of software, the blocks represent computer-executable instructions that, when executed by one or more processors, perform the recited operations. Generally, computer-executable instructions may include routines, programs, objects, components, data structures, and the like that perform particular functions or implement particular abstract data types. The order in which the operations are described is not intended to be construed as a limitation, and any number of the described blocks can be combined in any order and/or in parallel to implement the process. For discussion purposes, the processes are described with reference to the device 100 shown in FIG. 1.

At block 1702, the device 100 may measure a circumference of the limb at a repeatable home location over a period of time as an indicator of interstitial fluid volume in the limb. In some embodiments, the device 100 may be applied to a limb of a human subject and allowed to move to settle at the repeatable home location on the limb. In one or more embodiments, the repeatable home location may be the minimum circumference of the limb, where the strap tension balances the interstitial fluid pressure to enable the device to perform the measurements described above.

At block 1704, the device 100 may generate a waveform showing current circumference data derived from the measured circumference over time. In some embodiments, the circumference measurements may be taken periodically (e.g., once per day), intermittently (e.g., initiated manually), or continuously. In some embodiments, some or all of the data processing and waveform generation may be performed off-device, such as by a remote computing device.

At block 1706, the device 100 may compare the waveform with a waveform of baseline circumference data for the subject at the repeatable home location. The difference in waveforms may be interpreted by the device 105, transmitted to an off-device or remote location, such as a doctor's office, mobile device, portable computer, and/or the like, or interpreted by a human.

At block 1708, the device 100 may output a result of the comparing with an indication exposed by the comparing. For example, if the waveform of current circumference measurements shows an increase over the subject's baseline waveform, an alert may be displayed on the measurement assembly 105, transmitted to a mobile device or more remote endpoint. Additionally, or in the alternative, an instruction may be output to, e.g., advise the subject of changes to be made in diet or exercise, to revisit or make specific adjustments to treatment (including medicine dosage adjustments), and/or the like.

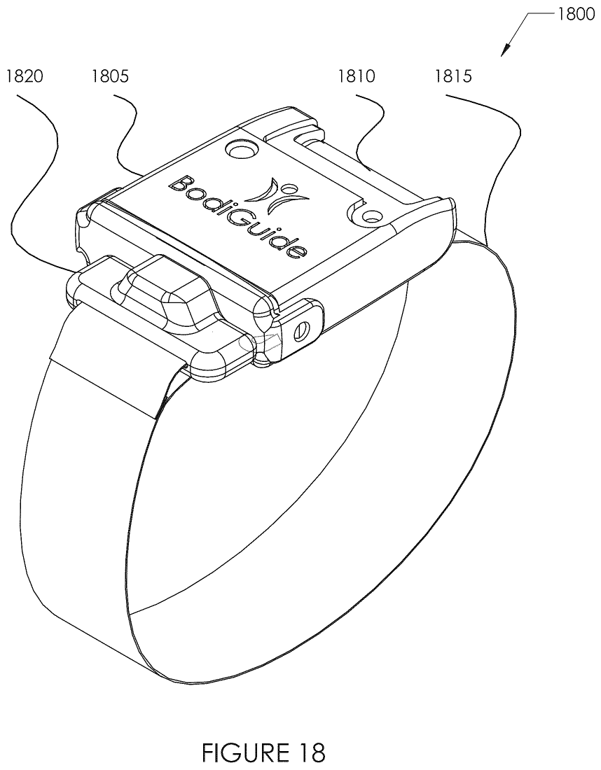

FIGS. 18-26 illustrate an example of another device. Aspects of the device that are similar to those of the device 100 are shown with like reference numbers, differing by the initial characters that indicate the figure in which the same are illustrated (e.g., the device 1800 illustrated in FIG. 18 may correspond to the device 100 shown in FIG. 1).

FIG. 18 shows an isometric view of a device 1800 as another example that can be configured to monitor changes in volume and properties of human interstitial fluid. The device 1800 may include, among other components, a device assembly comprising a measurement assembly 1805, a winder cassette 1810, a strap 1815 that is configured to be placed around the limb of the subject, and a clasp 1820. As with the device 100, the subject may wear the device 1800 around the lower arm (i.e., below the elbow) or lower leg (i.e., below the knee), although the teachings herein may apply to other body parts, especially body parts that have axial portions, and thus should not be considered limited except by the type of needed data and where the data might be obtained.

The measurement assembly 1805 may house an electronics subassembly to obtain data and communicate the data or information based on the data. The electronics subassembly, described below, may comprise sensors and associated electronics that, in some embodiments, may perform analysis of sensor data and output results of the analysis and/or recommendations or instructions based on those results. In some embodiments, the electronics subassembly may include a sensor portion comprising one or more magnetic sensors situated to be opposite one or more corresponding magnets on the winder cassette 1810, as described more fully below.

The winder cassette 1810 cooperates with the strap 1815 generally to enable a loose-tensioned but snug fit to the limb, even in the presence of severe swelling. As with the device 100, the device 1800 does not ordinarily fit tight to the limb, even in the presence of severe swelling. To this end, the strap 1815 and winder cassette 1810 are constructed so that the strap 1815 may extend and retract as the limb swells and contracts, enabling an essentially constant force to be applied to the limb. In some embodiments, the winder cassette 1810 combined with the strap 1815 and clasp 1820 may be a replaceable component that a user can change out easily if it were to become soiled or broken.

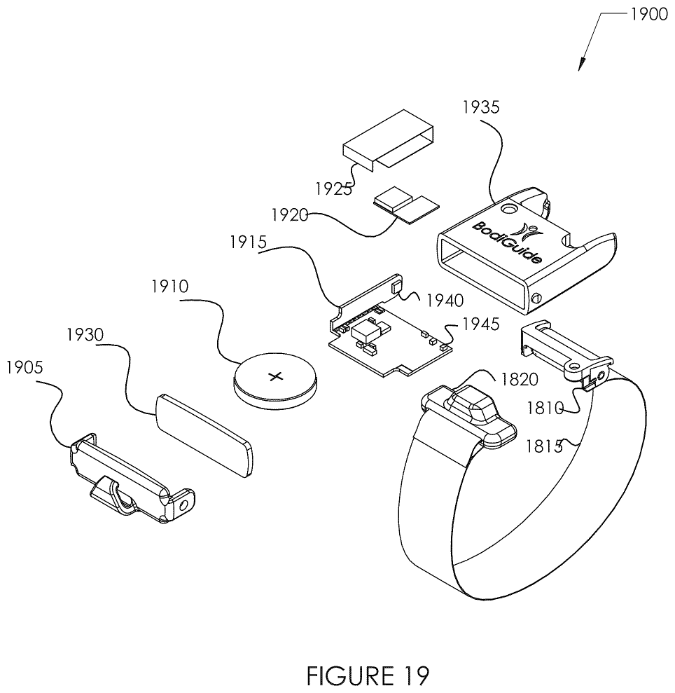

FIG. 19 shows an exploded view of an example measurement assembly 1805, winder cassette 1810, strap 1815, and clasp 1820 of a device 1900 corresponding to the example illustrated in FIG. 18. An enclosure 1935 and cover 1905 are configured to house a battery 1910; an electronics subassembly 1915 which includes a radio module 1920, an angle magnetic sensor 1940, and one or more strap-size magnetic sensor(s) 1945; an insulator 1925, and a gasket 1930. The measurement assembly 1805 may be substantially fluid-tight to prevent fluid ingress.

The battery 1910 may be similar to the battery 210. In some examples, the battery 1910 may be captured between the electronics subassembly 1915 and the top of the enclosure 1935. In some embodiments, another portable power source may be used, such as a rechargeable battery, fuel cell, storage capacitor, energy harvested from the patient, energy harvested from the environment, and the like.

The insulator 1925 may be an electrical insulator, and so can be positioned to insulate the battery 1910 from the electronics subassembly 1915 so that the battery 1910 can be changed without contacting any components or wiring on the electronics subassembly 1915 or the electronics board substrate. In some embodiments, the insulator 1925 may be stamped or printed with configuration information, such as version, for the device 1900 or any component thereof.

Similar to the electronics subassembly 215, the electronics subassembly 1915 may be a printed circuit board and include electronic components that control and carry out one or more of sensing of limb circumference. The limb circumference may be used by an on-board control system or transmitted to a remote computing device running an algorithm on data from the sensed limb circumference, outputting messages, and generating graphic results showing measurements over time that can be interpreted by a medical professional (for example) to give insight into the current condition of a patient wearing the device 1900. In some embodiments, the electronic components may include one or more magnetic sensors such as magnetic sensors 1940, 1945, one or more optical sensors, one or more processors, memory, and an accelerometer. The memory may store instructions that, when executed by the one or more processors, cause the one or more processors to carry out various operations described herein. In at least some embodiments, the magnetic sensors may detect limb circumference. In at least some embodiments, the accelerometer may detect patient orientation and motion and output data that may be used by an on-board control system or transmitted to a remote computing device to determine a level of activity of the patient. For example, the device, by information received from the accelerometer, may detect, accumulate, store, and/or transmit accelerometer measurements at a sufficiently high frequency and associate the information with circumference readings to provide an adequate representation of gravity effects and activity on the limb during the time between circumference measurements.

Like the radio module 220, the radio module 1920 may transmit a digital or analog signal (e.g., containing patient motion information) to an external device. For example, the signal may be transmitted to a nearby computer, smartphone, tablet, or the like which has processing power to receive the signal, analyze the data provided by the signal, output an instruction or command, and/or relay the signal to a remote computing device such as a server, computer, or database (at a doctor's office or data center, for example).

The gasket 1930 is configured and positioned to seal the enclosure 1935 and prevent dust or water intrusion that might harm sensitive components inside. For example, the gasket 1930 may be positioned between the enclosure 1935 and the cover 1905. The gasket 1930 comprises, without limitation, an insulative material suitable for its purpose. In at least one embodiment, the gasket 1930 may be solid rather than ring-like as disclosed in the example of the gasket 230. With the pictured gasket 1930, the gasket 1930 and cover 1905 may have flat surfaces and generous tolerance to mate with the opening of the enclosure 1935.

Like the measurement module 105, the measurement assembly 1805 may be constructed such that the insulator 1925 and electronics subassembly 1915 which includes the radio module 1920 are inserted into enclosure 1935. The cover 1905 may be joined together with the gasket 1930 positioned where the cover 1905 meets the enclosure 1935 by a snap fit, to facilitate easy removal of the cover 1905 for battery 1910 replacement. In this example, the cover 1905 may snap into the enclosure 1935, compressing the gasket 1930 which seals the interior of the enclosure 1935 from dust or water intrusion. Then, the winder cassette 1810 may be fitted to the measurement assembly 1805, by means of detents bringing together the measurement assembly 1805 and the winder cassette 1810, including the strap 1815.

The distal end of the strap 1815 is threaded through the body of the clasp 1820 and secured by adhering the strap 1815 to itself by heat joining with adhesive material the end to the body of the strap 1815. But it should be understood that many other methods of joinery such as strap 1815 material bonding to itself, the clasp 1820, impinging, and similar means are within the scope of this disclosure.

Similar to the clasp 120, the clasp 1820 can be joined to the electronics subassembly 1915 by latch features that engage geometry of the cover 1905 in the electronics subassembly 1915 in order to encircle the limb.

As in the case of the winder cassette 110, the winder cassette 1910 may be constructed to accommodate different length straps 1915, allowing the device 1900 to accommodate a broad range of application from a small wrist to a substantially swollen leg, for example.

In some embodiments, the enclosure 1935, cover 1905, and/or clasp 1820 may be 3D printed by the masked stereolithography (MSLA) process from materials such as Siraya Tech Blu resin and Siraya Tech Blu mecha nylon resin. The gasket 1930 may comprise Poron (polyurethane foam). The strap 1815 is generally flexible and inelastic, and may be made of a biocompatible, porous material for patient comfort. As one example, the material can be an open weave, 80 threads per inch polyester with fused edges, 0.008″ Teslin (PPG, Barberton, OH) or Tyvek (Wilmington, DL) or a nonwoven open fabric such as embroidery stabilizer.

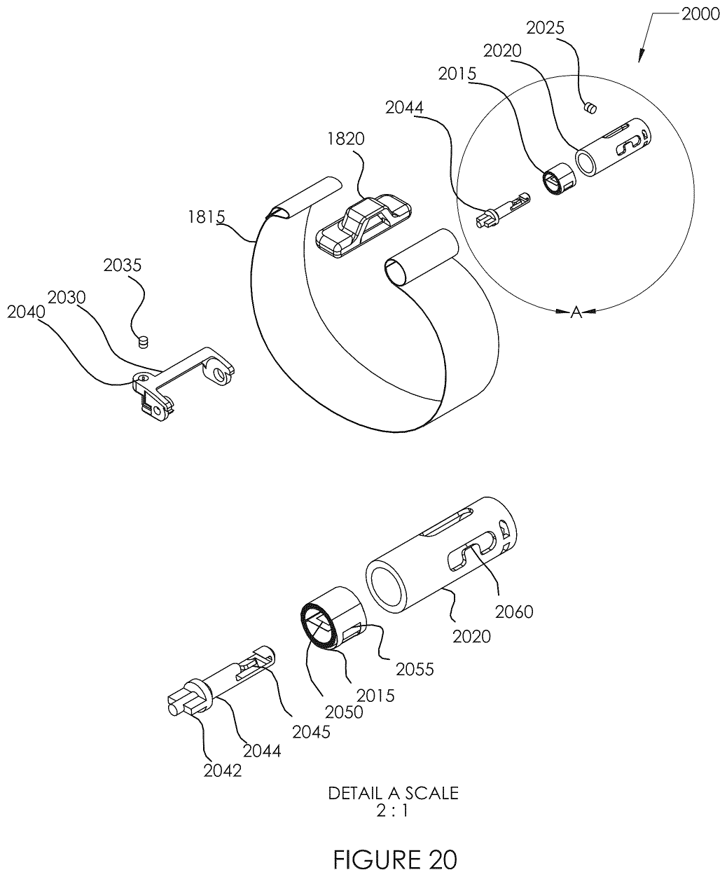

FIG. 20 shows an exploded view of the winder cassette 1810, strap 1815, and clasp 1820 that remains when the measurement assembly 1805 is removed in some embodiments. The winder cassette 1810 may comprise a winder cassette frame 2030, a spring 2015, and a spool 2020. The winder cassette frame 2030 may include a capture feature 2040 to receive the strap-size magnet 2035. The winder cassette frame 2030 may be configured to receive a drive pin 2044. The drive pin 2044 may include a drive pin feature 2042 (e.g., a projection or configuration to receive a projection) that is configured to be joined to a corresponding feature (such as a configuration to receive the projection of the drive pin feature 2042 or a projection corresponding to the receiving configuration of the drive pin feature 2042) in the winder cassette frame 2030. The spool 2020, which may be cylindrical, includes an angle magnet 2025. The strap 1815 may wrap around the spool 2020 when installed in the winder cassette frame 2030 as described below. In some embodiments, the winder cassette 1810, strap 1815, and/or clasp 1820, individually or as a combination that includes any two or all three, may be replaceable.

The spring 2015 may be a helical metal foil, at one portion of which is provided a hole 2050 that mates with a spring coupler, formed by a protrusion 2045 on the drive pin 2044 shown in FIG. 20, when the drive pin 2044 is inserted into the coiled spring 2015. Another portion of the spring 2015 may have a hole 2055 that hooks onto another spring coupler, formed of a protrusion 2060 in the spool 2020 shown in FIG. 20, to wind the spring 2015 and provide tension when the strap 1815 is extended. The spring 2015 may provide a substantially constant spring force load (for example, approximately 10-40 grams) sufficient to snug the strap 1815 to the limb without unnecessary, uncomfortable pressure.

The strap 1815 is attached and wound around the spool 2020. The angle magnet 2025 may be pressed or otherwise fitted into an opening, gap, or recess of the spool 2020 such that it opposes the angle magnetic sensor 1940 on the electronics subassembly 1915. The strap-size magnet 2035 may be pressed or otherwise fitted into an opening, gap, or recess of the winder cassette frame 2030 such that it opposes one or more of the strap-size magnetic sensor(s) 1945 on the electronics subassembly 1915 of the measurement assembly 1805. The magnetic sensors 1940 and 1945 are configured and positioned to sense the magnetic fields of the magnets 2025 and 2035 through the enclosure 1935.

The action of increasing and decreasing the length of the strap 1815 as the limb expands and contracts may be accomplished with a tensioning mechanism comprising the winder cassette frame 2030 and spring 2015 within the spool 2020 that may house a portion of the length of the strap 1815 that is wrapped around the spool 2020. In some embodiments, the spring 2015 may be a constant tension spring. As the limb expands, the strap 1815 unwinds, increasing the length of the strap 1815 to accommodate the increased circumference of the limb while maintaining a constant tension of the strap 1815 around the limb by a constant force applied by the spring 2015. The tension of the strap 1815 may be matched to the interstitial fluid pressure and elasticity of the skin such that the device expands and contracts without creating more than a negligible indentation in the limb. In this regard, and in conjunction with the other embodiments described herein, it is understood that constancy of the force and tension need not be exact but are within a reasonable tolerance that enables the device to perform its function of measuring limb circumference, and particularly differences thereof relative to a baseline or other reference, in accordance with the principles outlined in this disclosure.

The angle magnet 2025 fitted in spool 2020 may be sensed by the angle magnetic sensor 1940 such that as the winding and unwinding of the strap 1815 causes the spool 2020 to rotate, the degree or rotation of the spool 2020 may be recognized by a signal output by the angle magnetic sensor 1940 and received at the electronics assembly 1915, which is electrically connected to the angle magnetic sensor 1940.

The angle magnet sensor 1940 may be made up of a plurality of resistive elements that are arranged to output a variable set of signal strength voltages. In at least one embodiment, these correspond to the sine and cosine of the degree of rotation of the magnet 2025 field in relation to the magnetic sensor 1940. This can be accomplished by a full bridge sensor with an underlying spintronic technology (NVE corporation, Eden Prairie, MN produces suitable sensors at this time) as this produces a very low power construction that is relatively insensitive to distances and axial misalignment between the magnet 2025 and the angle magnet sensor 1940. As the diameter of spool 2020 defines a known circumference by the formula C=Pi×D, the diameter can be converted to precise changes in length measurements as the strap 1815 is expanded and contracted. The diameter of the spool 2020 may be determine/calibrated in manufacturing for each device.

In addition to measuring the degree of rotation of magnet 2025, the circuit retains information about rotation history. In this example, quadrature detection may be implemented in software or hardware to track the current revolution or number of revolutions. In at least one embodiment, quadrature detection may be accomplished by use of a comparator circuit that is connected to interrupt functionality on the processor. But in other implementations dedicated circuit counting and logic components or integrated functionality within the processor itself may serve this function. As the strap 1815 wraps around the spool 2020 for greater than 360 degrees, a count of rotations is maintained. The count of rotations in conjunction with the current angle measurement enables the software to calculate the total number of degrees of rotation. The diameter of the spool 2020 coupled with the total number of degrees of rotation in conjunction with the manufacturing strap 1815 length in the unexercised spring 1815 condition determines the total circumference measurement.

As described above in the discussion of FIG. 3, there may be an additional consideration to calculating total length. As suggested above, the strap 1815 as it wraps around the spool 2020 changes the effective diameter of the wrap and the circumference around the spool 2020 as layers stack on top of one another. To account for this condition it is possible to recognize the rotation history and manufacturing design of the winder cassette 1810 and apply a change in the diameter consistent with the thickness of the strap 1815 and the number of wraps of strap material about the spool 2020.

The circumference of the limb is equal to the sum of the unwound strap 1815 length at a home or retracted position, plus the length of the measurement assembly 1805 body and clasp 1820, plus the “variable length” of the strap 1815 that is wound and unwound from the spool 2020 as the limb expands and contracts at the minimum circumference or repeatable location. The variable length of the strap 1815 is equal to the proportional rotation corresponding to the current degree of rotation of the spool 2020 from the home position (defined as 0 degrees) plus the number of full rotations of the spool from the home position, multiplied by the spool circumference (i.e., the spool diameter multiplied by PI), considering the wound portion of the strap still on the spool. The proportional rotation current angle measured as the number of revolutions that has occurred from the home position may be computed from the arctangent of the sine/cosine that is provided by the magnetic sensor/magnet relationship and quadrant in which it occurs. The home position is captured when the strap is installed at the home position. The number of full rotations may be determined using quadrature detection (considering each 90 degrees of rotation measured from 0 degrees as one quadrant), which in this example may be detecting the number of times the spool has passed from quadrant 4 to quadrant 1 (see figure below—The Quadrature Plot).

The length of the strap 1815 when the spring 2015 is in the relaxed and unexercised condition is controlled when the winder cassette 1810 is manufactured. If the degree of rotation of magnet 2025 is consistent with a reference that corresponds to the manufactured relaxed orientation and the rotation history shows no additional winding, and there is no dithering of the angle from being worn, it may be assumed that the device 1900 is not being worn. In some embodiments, the spring 2015 may retract the strap 1815 to the home location, which can be read by the software that is executed to interpret the signals representing the detected angle of spool 2020, with an indicator or message output. The detection of these conditions also can be used to indicate whether a device is being worn. The data from the device 1900 in the “not worn” condition may be ignored in some embodiments in evaluating the condition of the wearer. Further, if this condition persists, the patient may be contacted or examined regarding any issues with wearing the device 1900.

As for the device 100, the device 1900 may accommodate a range of limb size in at least two ways, namely by the substantial spooling of the strap 1815 and/or by changing the total length of the strap to create winder cassettes 1810 of various sizes.

This construction may achieve at least two types of measurement. The first is a relative measurement quantifying only the changes in circumference associated with the change in spool from its home position as a result of the expansion and contraction of the limb The second type of measurement is an absolute measurement that can determine the total circumference of the limb by combining the relative measurement, length of the electronic assembly 1805, clasp 1820, and unexercised length of the strap 1815.

In the current example, the strap-size magnet 2025 in the winder cassette 1810 may communicate with one or more strap-size magnetic sensor(s) 1945 in the electronics subassembly 1915. Several strap-size magnetic sensors 1945 may be positioned so as to recognize a plurality of strap sizes by the relative position and orientation of the magnetic field generated by the strap-size magnet 2035. In at least one embodiment, a single strap-size magnet 2035 may be used to recognize as many as four strap sizes.

The strap-size magnetic sensor(s) 1945 can recognize the presence and/or the orientation of the strap-size magnet 2035 in the winder cassette 1810. In some embodiments, two strap-size magnetic sensors 1945, such as Hall effect sensors, can each output a signal in the presence of a north pole field or a different signal in the presence of a south pole field. By the arrangement of the relative position of the strap-size magnetic sensors 1945 and the orientation of the strap-size magnet 2035 in the winder cassette 1810, it is possible to detect and communicate five orientation conditions. For example, when no magnetic field is sensed, the winder cassette 1810 is determined (e.g., decoded) as not present; when the magnetic field axis is perpendicular to the circuit board supporting the strap-size magnetic sensors 1945, a north-north or south-south condition may be sensed; and when the magnetic field axis is parallel to the axis along the sensors 1945, a north-south or a south-north condition may be sensed. Sensing the presence or absence of the winder cassette 1810 with the strap-size magnet 2035+strap-size magnetic sensor 1945 coupling may allow for the recognition of a removal and replacement event. In addition, sensing the orientation of the strap-size magnet 2035 facilitates recognition of a size of the winder cassette 1810 such as small, medium, large, and extra-large. The winder cassettes 1810 of different strap 1815 lengths may be built with the strap-size magnet 2035 oriented such that the field presented to the strap-size magnetic sensor(s) 1945 in either intensity or field orientation can be read by the circuit and interpreted to communicate the size of the winder cassette 1810 attached to the measurement assembly 1805.

In some embodiments, the winder cassette frame 2030 and spool 2020 are 3D printed by the masked stereolithography (MSLA) process from materials such as Siraya Tech Blu resin and Siraya Tech Blu mecha nylon resin. The spring 2015 may be 125 millimeters long by 10 millimeters width by 0.025 millimeters (0.001 inch) full hard stainless steel shim stock (Precision Brands, Downers Grove, IL). The angle magnetic sensor 1940 may be a giant magneto resistor angle sensor such as AAT101-10E Full Bridge Angle Sensor by NVE (Eden Prairie, MN). The strap-size magnetic sensor(s) 1945 may be dual output unipolar hall effect switches such as AH1389 by Diodes Inc (Plano, TX). The magnets 2025 and 2035 are neodymium available from various suppliers.

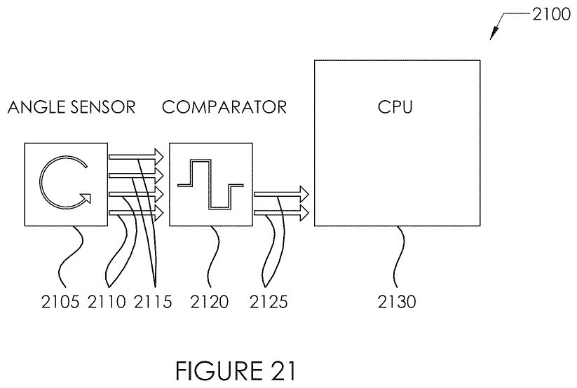

FIG. 21 illustrates an example of a digital circuit 2100 that may be implemented to perform quadrature decoding including counting revolutions (a digital pass through a 0 degree reference angle) detection in software at a fraction of the power used by some microprocessor quadrature decoder circuits. In such microprocessor quadrature detection circuits, interrupts may be fed into a microprocessor with quadrature detection that counts rotations, but this requires the microprocessor to remain awake which uses power. The digital circuit 2100 makes use of an interrupt in the context of reduced power usage, combining the interrupt with the analog angle measurement to keep track of quadrants and therefore count rotations accurately while accounting for dithering across a boundary between two adjacent quadrants. It should be noted that the digital circuit 2100 and/or measurement techniques described in its respect can be implemented with other embodiments described herein.

The digital circuit 2100 can trigger an interrupt that can wake the CPU or trigger additional software components to perform one or more of the actions described herein. In one example, the rate of interrupts associated with a rapid extension or retraction of the strap may be used to wake up the processor. In some embodiments, an angle sensor 2105 may produce electrical values corresponding to the sine 2110 and cosine 2115 of the angle of the magnetic field orientation to which it is exposed by the angle magnet 2025. A comparator 2120 simplifies the waveforms from the angle sensor 2105 (corresponding to the angle magnet sensor 1940) and determines the 0 and 180 degree crossing point signals 2125 of the waveform output (corresponding to the boundaries between quadrants Q4-Q1 and Q2-Q3, respectively). These crossing point signals 2125 may be electrically connected to the CPU 2130 input pins and may be used by the device 1900 as interrupts to the CPU 2130. These interrupts 2125 can be used to wake the CPU 2130 from a sleeping state to an active state, and can also be used to trigger a communication event such as a Bluetooth advertising condition or other software components.

In some embodiments, these crossing point signals 2125 can also be used in a low-power hybrid digital-analog approach to maintaining an accurate count of rotations when “dithering” might occur, such as when crossing the 360 to 0 boundary where the circuit detects or determines that a full rotation has occurred.

The following figure illustrates the method for correctly maintaining the rotation count.

Consider the small range between −1 and +1 deg from the reference 0 and call left of 0 West region and right of 0 East region. In this example, the digital 0-degree interrupt sensed region may be called the Rotation Count Region and the analog sensed region the Angle Region. In this range so close to 0, one or the other of the analog (Angle) or digital interrupt (Rotation Count) sensing may not agree. That is, measuring the angle per se may locate the angle in Q4 west of 0 and the digital interrupt detecting or determining the number of rotations (i.e., number of times passing the reference 0 crossing point) may be slightly off and locate the interrupt in Q1 east of 0. If we consider the Rotation Count Region to be correct, then an angle correction should be made. In one example, we can:
1. Keep track of the region via interrupting at both 0 and 180 so that we know the current region the Rotation Count is in.
2. Define the angle's region by 0-->1 deg=east and 359-->0=west.
3. Within this ±1 degrees, if the region determinations don't match, then add or subtract 360 degrees from the Angle measurement in order to match the Rotation Count Region, which has been assumed correct.
4. If the Rotation Count Region and Angle Region match, use the Angle measurement as is.

As mentioned, the rate of Rotation Count interrupts may be used to go into advertising mode. More specifically, the low-power detection of a rotation crossing combined with the time over which the crossing occurs may be used as a trigger to enter Bluetooth advertising mode, similar communication protocols, or other variable sections of software. In this way, the angle sensor interrupt can be used to perform both rotation counting and quickly extending or releasing the strap snapping the strap to go into advertising.

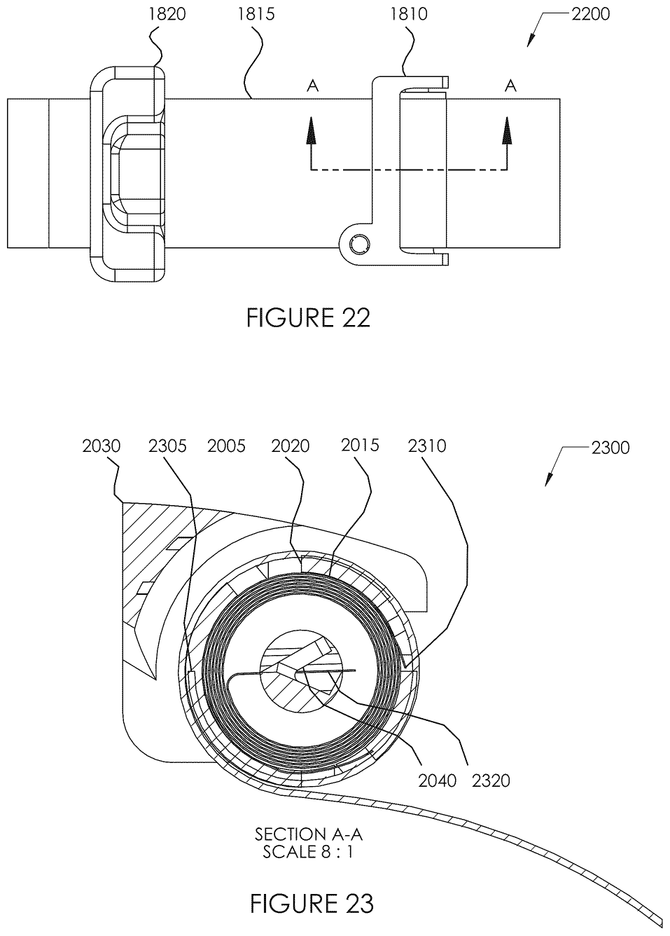

FIG. 22 is a top view 2200 of the winder cassette 1810 relative to the strap 1815 and clasp 1820. From this view, a partial cross section A-A of the winder cassette 1810 is located.

FIG. 23 is the partial cross section view A-A in FIG. 22 of the winder cassette 1810 in the assembled condition.

In some examples, the capture feature 2045 of the drive pin 2044 may fit within one or more tabs, grooves, or holes in a wall or walls or the winder cassette frame 2030. In some embodiments, one or both ends of the drive pin 2044 may be extruded from or fixed to the wall or walls. In such embodiments, the spring 2015 may be attached to the capture feature 2045 that mounts to the winder cassette frame 2030 by, e.g., inserting the end 2050 of the spring 2015 into a gap in the drive pin and wrapping or bending the spring 2015 around the drive pin 2044.

The coil spring 2015 may have a narrow wire or broad band-like construction, for example. To aid with fixing the spring 2015 to the capture feature 2040 of the winder cassette frame 2030, the end of the spring 2015 that is inserted into the gap may be wrapped or bent to wrap at least partially around the drive pin 2044. A bend configuration 2320 comprised of two bends in the spring 2015 may engage the capture feature 2040, as shown in FIG. 23. The distal end of the spring 2015 may be joined by adhesive 2310 to the spool 2020 or the protrusion 2060. The spool 2020 may in turn be attached to the strap 1815 by an adhesive 2305. No limitation on a particular type of fixing or adhesive should be inferred. This communicates the spring 2015 force from the capture feature 2040 in the winder cassette frame 2030 by the bend configuration 2320 of the spring 2015. The attachment of the winder cassette frame 2030 to the spring 2015, interlocked via the protrusion 2060 to the spool 2020, and in turn adhered 2305 to the strap 1815, tensions the assembly. The action of rotating the angle magnet 2025 opposite the angle magnetic sensor 1940 as the limb expands and contracts may be accomplished with this tensioning mechanism.

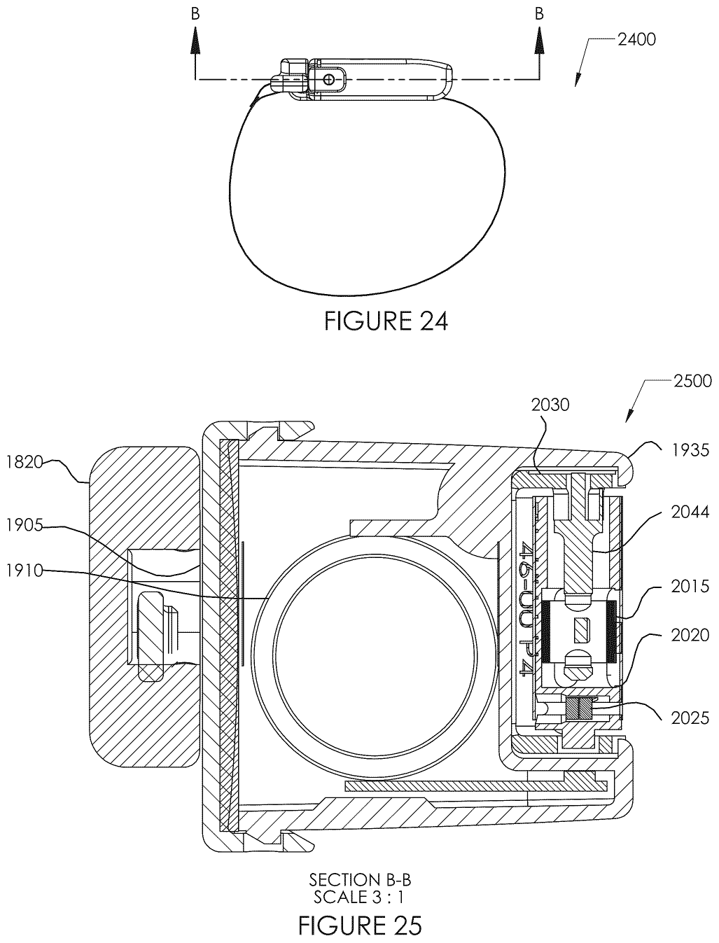

FIG. 24 is a side view 2400 of the device, which may correspond to the device 1900. In practice, the device 1900 may drift to its operative, repeatable home location on the limb by the influence of gravity and patient movement, as influenced by friction between the strap 1815 and the limb.

FIG. 25 is a detail section view 2500 through the measurement assembly 1805, winder cassette 1810, strap 1815, and clasp 1820 of the example device illustrated in FIG. 24, taken along line B-B. This illustrates the engaged use position of the clasp 1820 to the cover 1905 and the position of the battery 1910 in the enclosure 1935. A scale of 3:1 is indicated to give a sense of the size of the device, which may correspond to the device 1900. However, the scale will change depending on the size of the figure as presented on the page. That is, zooming in or out will affect the scale, and thus 3:1 should not be considered limiting.

On the right side of FIG. 25, the winder cassette 1810 is in its use position. The winder cassette frame 2030, spool 2020, spring 2015, angle magnet 2025, and drive pin 2044 work in conjunction to communicate the degree of rotation of the spool 2020 and the overall length of circumference measurement about the limb of the wearer, determined as described elsewhere herein.

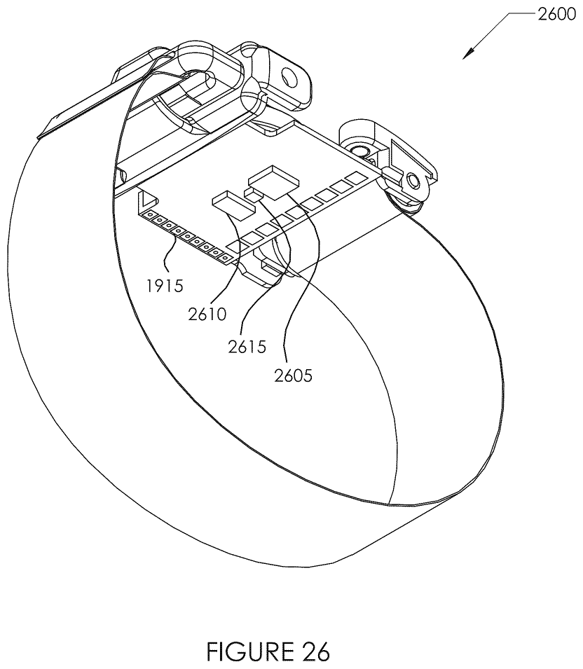

FIG. 26 is an isometric view 2600 of the device, which may correspond to the device 1800 illustrated in FIG. 18 with the enclosure of the measurement assembly removed. The view orientation shows the electronics that are in close proximity to the limb with the rear enclosure 1935 removed for illustrative purposes only. On the electronics subassembly 1915 there may be components for the sensing of heart rate, SPO2, NIBP and like physiological parameters. In the example shown an optical emitter 2615 that may emit light in the green, red and infrared wavelengths is placed centrally and in close proximity to the wearer. Light from this emitter component travels into the skin of the wearer. Some of this light is reflected back and is sensed by detector components 2605 and 2610. The returned light is sensed and evaluated by the software. The raw or evaluated measurements can then be transmitted to other computing devices.

In some embodiments, the device 1900 may use orientation information detected by the accelerometer to detect when the patient is in the horizontal position or resting. Resting heart rate is sampled. Variation in resting heart rate is known to change as a patient retains fluid. These heart rate readings can be used to increase confidence in the interpretation of circumference measurements.

Limb circumference measurements and limb orientation data may be taken continuously at a regular interval and processed to produce a personal Daily Swelling Pattern for the subject wearing the device. The Daily Swelling Pattern is characterized by a minimum limb circumference that occurs when the subject is lying down and at a maximum circumference after the subject has been in a vertical position such as standing or sitting for a period of time specific to that individual.

Trends in the fluid gain or loss can be computed for specified time periods such as days, weeks, or months. Fluid gain/loss and fluid gain/loss trends can be compared to threshold values to identify conditions of interest. The system takes actions specific to the condition of interest including sending messages and alerts to the user as well as support personnel such as family caregivers, chronic care management, and/or clinical personnel.

The rate at which fluid redistributes itself in the body on rising to a vertical orientation may be indicative of the viscosity of the interstitial fluid; changes in viscosity are known to be related to heart failure decompensation due to changes in protein levels in the interstitial fluid. This is typically evaluated by a physician pushing a finger firmly against the ankle of the patient and seeing if the “dent” produced rebounds quickly. If the dent is slow to rebound, the condition is described as pitting edema. This is a significant medical sign and useful diagnostic method in characterizing the condition of the patient.

The disclosed techniques can characterize the rate of change of the redistribution of interstitial fluid. By taking a plurality of measurements when the patient moves from a supine to upright orientation, typically in the morning, it is possible to track the time it takes for the redistribution of interstitial fluid associated with the change in direction of the gravitational force. This rate of change measurement is directly related to the viscosity of the interstitial fluid. Being able to recognize interstitial fluid viscosity and changes associated with it can further inform the medical practitioner or computed algorithm about changes in the disease state of the patient, as fluid viscosity offers insight to the underlying cause of fluid loading (e.g., change in protein levels in the interstitial fluid).

Although the subject matter has been described in language specific to structural features and/or methodological acts, it is to be understood that the subject matter defined in the appended claims is not necessarily limited to the specific features or acts described above. Rather, the specific features and acts described above are disclosed as an example forms of impending the claims.

What is claimed is:

1. A device to determine a level of interstitial swelling in a subject, comprising:
a winder cassette that includes:
a frame;
a capture feature supported by the frame, the capture feature including a drive pin and the drive pin having a first spring coupler;
a spring coupled to the capture feature via the first spring coupler; and
a spool supported by the frame and surrounding the spring, the spool supporting a first magnet and having a second spring coupler via which the spool is coupled to the spring; and
a measurement assembly that includes an electronics subassembly, wherein the electronics subassembly includes:
measurement components including a first magnetic sensor arranged to be magnetically couplable to the first magnet; one or more processors; memory; and executable instructions stored in the memory that, if executed by the one or more processors, cause the one or more processors to perform operations comprising:
determining a first measurement of a first magnetic coupling between the first magnet and the first magnetic sensor;
determining a first angle of a strap-wrapped spool based on the first measurement;
determining the number of rotations of the spool;
determining a length of strap unwrapped from the spool based on the first angle and number of rotations of the spool;
determining a first circumference representing the volume of interstitial fluid based on the length of strap unwrapped from the spool.

2. The device of claim 1, further comprising:
at least one interrupt circuit that includes a magnetic sensor and generates interrupts to one of the one or more processors,
wherein in response to the rate of interrupts exceeding a preset threshold, the one of the one or more processors enters a predefined state.

3. The device of claim 3, wherein the predefined state is a wake mode.

4. The device of claim 3, wherein the predefined state is an advertise mode.

5. The device of claim 1, further comprising;
a magnetic sensor configured to output at least two analog signals associated with an angular position of the spool, and
at least one interrupt circuit that includes a magnetic sensor,
wherein in response to detecting an interrupt associated with a full rotation of the spool, the number of rotations is confirmed using the angle of the spool determined from the angular position.

6. The device of claim 1, further comprising:
a second magnet supported by the frame,
wherein the measurement components further include a second magnetic sensor arranged to be magnetically couplable to the second magnet, and wherein the operations further comprise:
determining a polarity of a second magnetic coupling between the second magnet and the second magnetic sensor; and
determining a maximum extension of the strap based on the polarity.

7. The device of claim 1, wherein the first spring coupler comprises a protrusion configured to couple to a corresponding first aperture in the spring, and the second spring coupler is a protrusion configured to couple to a corresponding second aperture in the spring.

8. A computer-implemented method to determine an increase or decrease of interstitial volume in a subject, the method comprising:
via one or more magnetic sensors in a winder cassette that includes a frame; a capture feature supported by the frame, the capture feature including a magnet; a spring supported by the capture feature; and a spool supported by the frame and surrounding the spring, the spool supporting the magnet; and a measurement assembly that includes an electronics subassembly, wherein the electronics subassembly includes measurement components including a magnetic sensor arranged to be magnetically couplable to the magnet; one or more processors; memory; and executable instructions stored in the memory that, if executed by the one or more processors, cause the one or more processors to perform operations comprising:
determining a measurement of magnetic coupling between the magnet and the magnetic sensor;
determining a first diameter of a strap-wrapped spool based on the measurement, the first diameter including a diameter of the spool itself plus a wrapped thickness of the strap wrapped on the spool;
determining a first circumference of the wrapped spool based on the first diameter;
determining a length of the wrapped strap around the spool based on the first circumference, the length being the full length of the strap minus the amount of the strap wrapped around the spool; and
determining a level of interstitial swelling based on the length.

9. The method of claim 8, further comprising:
determining a change in the circumference of the strap on the wrapped spool beyond a preset length threshold; and
in response to determining the change in the circumference beyond the preset length threshold, triggering at least one of the one or more processors to enter a predefined state.

10. The method of claim 9, wherein the predefined state is a wake mode.

11. The method of claim 8, further comprising:
determining a rate of interrupts to one of the one or more processors; and
in response the rate of interrupts exceeding a preset threshold, triggering the one of the one or more processors to enter a predefined state.

12. The method of claim 11, wherein the predefined state is a wake mode.

13. The method of claim 8, further comprising:
determining a change in the circumference of the strap on the wrapped spool beyond a preset acceleration threshold; and
in response to determining the change in the circumference beyond the preset acceleration threshold, triggering the one of the one or more processors to enter a predefined state.

14. The method of claim 13, wherein the predefined state is a wake mode.

15. The method of claim 8, further comprising:
integrating the determining of the level of interstitial swelling with automatic real-time control of treatment of the patient.

16. One or more non-transitory computer-readable media containing executable instructions that, if executed by one or more processors, cause the one or more processors to perform operations to determine an increase or decrease of interstitial volume in a subject via one or more magnetic sensors in a winder cassette that includes a frame; a capture feature supported by the frame, the capture feature including a magnet; a spring supported by the capture feature; and a spool supported by the frame and surrounding the spring, the spool supporting the magnet; and a measurement assembly that includes an electronics subassembly, wherein the electronics subassembly includes measurement components including a magnetic sensor arranged to be magnetically couplable to the magnet; the one or more processors; and memory, the operations comprising:
determining a first measurement of a first magnetic coupling between the first magnet and the first magnetic sensor;
determining a first angle of the strap-wrapped spool based on the first measurement;
determining the number of rotations of the spool;
determining a length of strap unwrapped from the spool based on the first angle and number of rotations of the spool; and
determining a first circumference representing the volume of interstitial fluid in the subject based on the length of strap unwrapped from the spool.

17. The one or more non-transitory computer-readable media of claim 16, the electronics subassembly further including at least one interrupt circuit that includes a magnetic sensor and generates interrupts to one of the one or more processors, the operations further comprising:
determining a rate of the interrupts; and
in response to the rate of interrupts exceeding a preset threshold, triggering the one of the one or more processors to enter a predefined state.

18. The one or more non-transitory computer-readable media of claim 17, wherein the predefined state is a wake mode.

19. The one or more non-transitory computer-readable media of claim 17, wherein the predefined state is an advertise mode.

20. The one or more non-transitory computer-readable media of claim 16, the operations further comprising:
integrating the determining of the level of interstitial swelling with automatic real-time control of treatment of the patient.
